(19) United States
(12) Patent Application Publication
(10) Pub. No.: US 2012/0142069 A1
(43) Pub. Date: Jun. 7, 2012

(54) INTERPENETRATING BIOMATERIAL MATRICES AND USES THEREOF

(75) Inventors: Lonnie D. Shea, Chicago, IL (US); Teresa K. Woodruff, Chicago, IL (US); Ariella Shikanov, Chicago, IL (US)

(73) Assignee: NORTHWESTERN UNIVERSITY, Evanston, IL (US)

(21) Appl. No.: 13/382,709

(22) PCT Filed: Jul. 8, 2010
(86) PCT No.: PCT/US2010/041388

§ 371 Date: Feb. 21, 2012

Related U.S. Application Data
(60) Provisional application No. 61/223,808, filed on Jul. 8, 2009.

(51) Int. Cl. C12N 11/10; C12N 9/74

(52) U.S. Cl. 435/178; 435/214

(57) ABSTRACT

The present invention relates to matrices (e.g., fibrin-alginate matrices; fibrin-alginate-matrigel matrices) for culture of cells, organs (e.g., ovary or fragment thereof), cells and cell aggregates (e.g., ovarian follicles, embryoid bodies), and tissues. In some embodiments, protease inhibitors e.g., aprotinin) are used to prevent the degradation of fibrin.

### CROSS REFERENCE TO RELATED APPLICATIONS

This Application claims priority to U.S. Provisional Patent Application Ser. No. 61/223,808, filed Jul. 8, 2009, hereby incorporated by reference in its entirety.

This invention was made with government support under Grant Number U54 HD041857awarded by the National Institutes of Health (National Institute of Child Health and Human Development). The government has certain rights in the invention.

### FIELD OF THE INVENTION

The present invention relates to matrices (e.g., fibrin-alginate matrices; fibrin-alginate-matrigel matrices) for culture of cells, organs (e.g., ovary or fragment thereof), cells and cell aggregates (e.g., ovarian follicles, embryoid bodies), and tissues. In some embodiments, protease inhibitors (e.g., aprotinin) are used to prevent the degradation of fibrin.

### BACKGROUND OF THE INVENTION

Loss of female reproductive capacity is a common cause of concern for young female cancer patients who are treated with gonadally toxic chemotherapeutics, radiation or surgery. Hormone stimulation followed by oocyte cryopreservation and/or in vitro fertilization (IVF) and embryo cryopreservation is the most common approach for preserving fertility in female cancer patients prior to initiating chemotherapy or radiation therapy (Jeruss et al. (2009) New England J Med. 360(9):902-1 1; Oktay et al. (2003) Human Reprod. 18:90-95; Rao et al. (2004) Lancet 363:1829-30; Juretzka et al. (2005) Fertil. Steril. 83:1041; Oktay (2005) Lancet Oncol. 6:192-3; Lee et al.(2006) J Clin Oncol. 24:2917-31; Smitz et al. (2010) Hum. Reprod. Update 16:395-414; West et al. (2009) Pediatr. Blood Cancer 53:289-295; each herein incorporated by reference in its entirety). However, hormone stimulation requires a delay in cancer treatment, may be contraindicated in patients with hormone-sensitive malignancies, and is not an option for adolescent and prepubertal girls. As the number of young cancer survivors increases, there is a need for fertility preservation options that do not require hormonal stimulation or delay necessary cancer treatment.

A potential alternative strategy for fertility preservation for these patients involves ovarian tissue cryopreservation; at a later date, the thawed tissue could be used in orthotopic transplantation, or immature follicles could be retrieved from the tissue for in vitro follicle growth, in vitro oocyte maturation (IVM), and fertilization (West et al. (2009 Pediatr. Blood Cancer 53:289-295; Demirtas et al. (2008) Reprod. Biomed. Online 17:520-523; each herein incorporated by reference in its entirety). While ovarian tissue transplantation has been successful, it carries a risk of reintroducing malignant cells. While in vitro follicle growth techniques have been successful in rodent species, there is need for improved methods for ovarian tissue culture of ovarian follicles (e.g., primary follicles, secondary follicles) of nonhuman primates and humans. In addition, there is need for improved methods for follicle culture in other species, including livestock and endangered animal species.

### SUMMARY OF THE INVENTION

The present invention relates to matrices (e.g., fibrin-alginate matrices; fibrin-alginate-matrigel matrices) for culture of cells, organs (e.g., ovary or fragment thereof), cells and cell aggregates (e.g., ovarian follicles, embryoid bodies), and tissues. In some embodiments, protease inhibitors (e.g., aprotinin) are used to prevent the degradation of fibrin.

Embodiments of the present invention provide matrices such as interpenetrating networks (e.g., comprising fibrin-alginate) for use in the culture of organized cell clusters. The compositions of methods of embodiments of the present invention find use in a variety of applications including, but not limited to, growth and maturation of ovarian follicles and oocytes (e.g., for use in in vitro fertilization) and growth of additional organized cell clusters. In some embodiments, methods of the present invention also find use as bioassays of follicular health (e.g., viability, metabolic activity, growth, and/or development of cultured follicles). In some embodiments, the degree and/or rate of matrix component (e.g., fibrin) degradation by a follicle cultured in a fibrin-alginate matrix finds use as a bioassay of follicular health (e.g., viability, metabolic activity, growth, and/or development of cultured follicles).

In certain embodiments, the present invention provides a method of culturing an organized cell cluster in vitro comprising providing a two-component interpenetrating network (IPN), providing the organized cell cluster, encapsulating the organized cell cluster in the two-component interpenetrating network, and culturing the encapsulated organized cell cluster in vitro. In some embodiments, the two-component interpenetrating network comprises fibrin and alginate. In some embodiments, the organized cell cluster a type such as an ovarian follicle, matrix-directed cardioprogenitor cells, embryoid bodies, or primary cell co-cultures. In some embodiments, the ovarian follicle is a type such as a primordial follicle, a primary follicle, a secondary follicle, a preantral follicle, or an antral follicle. In some embodiments, encapsulating occurs by introduction of the organized cell cluster into the two-component interpenetrating network, wherein the interpenetrating network is in a form such as a bead, a culture plate insert, a transwell, or a droplet. In some embodiments, the two-component interpenetrating network comprises a cross-linking agent. In some embodiments, the cross-linking agent is thrombin. In some embodiments, the two-component interpenetrating network comprises calcium chloride. In some embodiments, the alginate is present at a final concentration of 0.125%. In some embodiments, the fibrin is formed by fibrinogen. In some embodiments, the fibrinogen is present in the interpenetrating network at a final concentration of 12.5 mg/ml. In some embodiments, the thrombin is present at a final concentration such as 5 IU/mL, 50 IU/mL, or 500 IU/mL. In some embodiments, the interpenetrating network further comprises a protease inhibitor. Examples of protease inhibitors include but are not limited to aprotinin, 4-(2-aminoethyl)benzenesulfonyl fluoride (AEBSF), amastatin-HCl, alpha1-antichymotrypsin, antithrombin III, alpha1-antitrypsin, 4-aminophenylmethane sulfonyl-fluoride (APMSF), arphamenine A, arphamenine B, E-64, bestatin, CA-074, CA-074-Me, calpain inhibitor I, calpain inhibitor II, cathepsin inhibitor, chymostatin, diisopropylfluorophosphate (DFP), dipeptidylpeptidase IV inhibitor, diprotin A, E-64c, E-64d, E-64, ebelactone A, ebelactone B, EGTA, elastatinal, foroxymithine, hirudin, leuhistin, leupeptin, alpha2-macroglobulin, phenylmethylsulfonyl fluoride (PMSF), pepstatin A, phebestin, 1,10-phenanthroline, phosphoramidon, chymostatin, benzamidine HCl, antipain, epsilon-aminocaproic acid, N-ethylmaleimide, trypsin inhibitor, 1-chloro-3-tosylamido-7-amino-2-heptanone (TLCK), 1-chloro-3-tosylamido-4-phenyl-2-butanone (TPCK), trypsin inhibitor, and sodium EDTA. In some embodiments, the protease inhibitor comprises aprotinin. In some embodiments, the method further comprises subjecting the cultured, organized cell cluster to a process such as in vitro maturation or in vitro fertilization. In some embodiments, the interpenetrating network further comprises a proteinaceous extract of Engelbreth-Holm-Swarm mouse sarcoma. In some embodiments, the extract comprises Matrigel™ matrix (BD Biosciences, Bedford, Mass.). In some embodiments, the culturing is conducted in the presence of Follicle-Stimulating Hormone (FSH).

In certain embodiments, the present invention comprises a system for culturing an organized cell cluster in vitro, the system comprising an organized cell cluster and a two-component interpenetrating network.

In certain embodiments, the present invention comprises a kit for culturing an organized cell cluster in vitro, the kit comprising: fibrinogen, alginate, thrombin, and calcium. In some embodiments, kits comprise one or more of components such as media (e.g., maintenance media (e.g., αMEM), bovine serum albumin, growth factors (e.g., FSH, TGF (e.g., TGFβ1), EGF, bFGF, VEGF), fetuine, insulin, transferrin, and selenium. In some embodiments, kits may contain components such as alginate (e.g., pre-formed alginate) (e.g., alginate beads)), protease inhibitor (e.g., aprotinin), culture vessels (e.g., microwell plates, 96-well plates), and instructions for use.

In certain embodiments, matrices (e.g., interpenetrating networks, fibrin-alginate interpenetrating networks) formed by methods of the present invention have an initial storage modulus (G′) of less than 5; 5-50; 50-100; 100-200; 200-300; 300-500; 500 Pa or higher. In certain embodiments, the storage modulus of matrices of the present invention change over time (e.g., during the process of matrix formation; while in use for cell culturing) such that a final storage modulus is reached. In certain embodiments, the final storage modulus is less than 5; 5-50; 50-100; 100-200; 200-300; 300-500; 500 Pa or higher. In preferred embodiments, the storage modulus is initially high (e.g., preferably 100 Pa or higher; more preferably 200 Pa or higher; most preferably 250 Pa or higher) and decreases over time (e.g., during the course of use of the matrix for cell culture) to a lower storage modulus (e.g., preferably 100 Pa or lower; more preferably 75 Pa or lower; most preferably 50 Pa or lower). Additional embodiments are described herein.

### DESCRIPTION OF THE DRAWINGS

FIG. 1 shows rheometric characterization of gelation rate and gel mechanics. The storage modulus G′ (black symbols) and loss modulus G″ (grey symbols) during the crosslinking reaction (A) and amplitude sweep test (B) of IPNs with different concentrations of thrombin are indicated. For the amplitude sweep test, the gels were crosslinked for 10 min and then tested.

FIG. 2 shows SEM images of (A) fibrin gel 50 IU/mL thrombin, (B) FA-IPN with 5 IU/mL thrombin, (C) FA-IPN with 50 IU/mL thrombin, (D) FA-IPN with 500 IU/mL thrombin. The fibrin gel and FA-IPNs were prepared with 25 mg/mL fibrinogen. The scale bar is 3 μm.

FIG. 3 shows two-layered secondary follicle growth in FA-IPNs: (A-D) a representative encapsulated follicle at day 2 (A), 4 (B), 8 (C) and 12 (D); (E) fixed and H&E stained follicle after 12 day culture; growth curve over a 12-day culture period (F) and percent increase in follicle diameter relative to day 0 (G) in FA-IPNs with 5, 50 and 500 IU/mL thrombin.

FIG. 4 shows the degradation process of the fibrin, which results in a clearing ring in a matrix around the encapsulated growing follicle (black arrow). (A-D) Bright field images of the degradation ring on day 0 (A), day 2 (B), day 4 (C) and day 6 (D) during in vitro culture in FA-IPNs; (E) the distance from the follicle to the edge of the degradation ring during the culture in FA-IPNs with 5, 50 and 500 IU/mL thrombin; (F-G) H&E staining of the FA-IPN with degradation ring of the fibrin on day 2 (F) and day 3 (G). Scale bar 100 μm.

FIG. 5 shows follicles cultured in fibrin gel (25 mg/mL fibrinogen, 50 mg/mL thrombin). (A) Follicle cultured in fibrin gel on day 6 degraded the matrix around it and had support only on one side (FG: fibrin gel); (B) Granulosa cells in follicle cultured in a transwell at day 6 started to migrate away from the oocyte (white arrows); (C) Follicle cultured in fibrin gel in transwell reached 500 μm in diameter, but appeared flat similar to 2D culture (Oo:oocyte).

FIG. 6 shows steroid secretion profiles of two-layered secondary follicles cultured in FA-IPNs with 5, 50 and 500 IU/mL thrombin. Androstenedione (A), Estradiol (B) and Progesterone (C) increased as follicles developed in the culture, no significant difference was observed between the different conditions on day 12.

FIG. 7 shows two layered secondary follicles cultured for 12 days in FA-IPN. Follicles were matured in vitro and oocytes resumed meiosis and extruded the first polar body (dashed arrow), bright field image, 40× (A) and confocal image of the MII stage follicle with the spindle (B, C, spindle is pointed with solid arrows).

FIG. 8 shows a collection of small antral follicles from a luteal-phase baboon ovary. (A) Small antral follicles observed under a stereomicroscope (arrowheads). (B) H&E stained section of a luteal-phase baboon ovary with arrowheads indicating the location of small antral follicles on the border of the ovarian cortex and medulla. Bar=1 mm.

FIG. 9 shows small antral follicle COC oocyte status at baseline and after IVM. (A-C) OL-COC oocytes remained in the GV stage (black arrows) through 48 hours of IVM. (D-F) 1L-COC and (G-I) ML-COC oocytes displayed cumulus cell mucification within 24 hours and extruded the first polar body (F and I, arrows) by 48 hours. Bar=50 μm.

FIG. 10 shows small antral follicle oocyte spindle morphology and chromosome alignment. (A) Normal bipolar spindle/aligned chromosome; (B) Bipolar spindle/nonaligned chromosome; (C) Disarranged spindle/aligned chromosomes; (D) Severely disarranged or absent spindle/dispersed or absent chromosomes. (E) GV-stage oocytes developed to a mature oocyte (F) with a normal MII spindle and first polar body (FPB). DNA is stained with PI; α-tubulin was stained with Alexa Fluro 488. Bar=10 μm.

FIG. 11 shows characteristics of in vitro baboon embryo development. A total of 33 MII oocytes resulting from IVM of baboon small antral follicle COCs were fertilized by ICSI; by day 1, 8 had visible pronuclei (2PN); by day 2, 4 embryos had reached the 2-cell stage; and by day 4, 2 embryos had reached the morula stage.

FIG. 12 shows characteristics of in vitro cultured preantral baboon follicles. (A) Follicles cultured in the absence of FSH had a compact structure, whereas (B) follicles cultured in 100 mIU/ml FSH showed separation of cumulus cells from the oocyte after 10 days. (C) COCs recovered from the FAM beads after 10 days of culture in the absence of FSH had multiple cumulus layers, compared with (D) COCs recovered from follicles cultured in the presence of FSH. Bar=100 μm (A, B), 50 μm (C, D).

FIG. 13 shows follicle and oocyte size during in vitro preantral follicle culture. (A) Baboon preantral follicles grew continuously for 10 days in the presence of 10 or 100 mIU/ml FSH, or for 14 days in the absence of FSH. (B) After 14 days of culture without FSH, the average oocyte size increased from 95.0±0.5 μm to 105.6±2.1 μm, similar to the size of oocytes within small antral follicle COCs (in vivo; 104.6±1.0 μm). Different letters indicate statistically significant differences (P<0.05).

FIG. 14 shows baboon preantral follicle growth in FAM and IVM of recovered oocytes. (A) Preantral follicle (223 μm) isolated from the luteal-phase baboon ovarian cortex and encapsulated in FAM. (B) After 14-days' culture in the absence of FSH, the follicle developed to the small antral follicle stage (667 μm). (C) Compact COCs were recovered from the FAM culture beads for IVM. (D) Cumulus cells expanded after 24 hours' IVM. (E, F) The oocyte resumed meiosis and reached the MII stage within 48 hours, and showed normal spindle structure and the first polar body (FPB, black arrow). Bar=100 μm (A-D), 50 μm (E), 10 μm (F).

FIG. 15 shows representative photomicrographs of H&E-stained paraffin sections of whole ovaries before and after culture. (A) Control, uncultured 8-day-old mouse ovary. (B) Eight-day-old mouse ovary after 4 days of organ culture. (C) H&E staining of uncultured 8-day-old mouse ovary, which contains mainly primordial follicles with a few primary and secondary follicles. (D) H&E staining of 8-day-old mouse ovary after 4 days of organ culture. More secondary follicles were observed. (E) H&E staining of uncultured 12-day-old mouse ovary. (F) Follicle distribution in mouse ovaries before and after 4-day organ culture. 0, primordial follicle; 1, primary follicle; 2, secondary follicle; scale bars in A and B=150 μM; other scale bars=200 μM; letters indicate a statistically significant difference between groups (P <0.05).

FIG. 16 shows development and differentiation of representative secondary follicles cultured in vitro. (A) Secondary follicles with centrally located immature oocytes isolated from cultured ovarian tissues. (B, C) Follicles maintained their 3D structure with proliferation of granulosa cells, antrum formation (white arrowhead), and development of theca cell layers (black arrowhead) after 12 days of culture in 0.25% alginate or FA. (D) Follicle diameter in both culture systems increased significantly during the culture period. (E) Oocyte size increased significantly over the culture period. Statistically significant differences were observed between groups as indicated with different letters (P<0.05). Scale bar=100 04. (F, G) Average values of E₂ (F) and P (G) secretion were measured in conditioned culture media from secondary follicle cultures.

FIG. 17 shows meiotic and fertilization competence of oocytes from follicles cultured for 12 days in alginate (A-D) or FA (E-H), as assessed by in vitro maturation (IVM) and in vitro fertilization (IVF). CEOs isolated from antral follicles retrieved from alginate (A) or FA (E) culture systems were induced with hCG for 18 hours in vitro. (B, F) In both environments, cumulus cells around the oocytes expanded. (C, G) Oocytes resumed meiosis and extruded the first polar body (arrowhead). (D, H) Two-cell embryos were obtained by IVF of MII oocytes. Scale bar=50 04.

FIG. 18 shows that the degradation process of the fibrin appears as a clearing ring in a matrix around the encapsulated growing follicle. (A) Bright field images of the degradation ring on days 2, 4, 6, 10 and 12 without additional aprotinin in the culture media. (B) when 0.01 TIU/mL or 0.1 TIU/mL (C) of aprotinin were added to the culture media at days 0, 2 and 4.

FIG. 19 shows two-layered secondary follicle growth in FA-IPNs: growth curve over a 12-day culture period without aprotinin (green curve), with 0.01 TIU/mL aprotinin (blue curve) or 0.1 TIU/mL aprotinin (red curve) added on days 0, 2 and 4 of the culture.

FIG. 20 shows two layered—secondary follicles cultured for 12 days in FA-IPN without aprotinin or 0.01 TIU/mL aprotinin at days 0,2 and 4. The follicles were matured in vitro and oocytes resumed meiosis and extruded the first polar body (arrow), bright field image.

### DEFINITIONS

As used herein, the terms “matrix” and “matrices” refer to a network of materials (e.g., biomaterials) that may or may not contain chemical bonds between the individual components. In some embodiments, matrices are “interpenetrating networks” in which at least one polymer or individual component is synthesized or crosslinked in the presence of the other, either simultaneously or sequentially. In some embodiments, matrices are comprised of fibrin and alginate, crosslinked with a suitable crosslinking agent.

The term “sample” is used in its broadest sense. On the one hand it is meant to include a specimen or culture. On the other hand, it is meant to include both biological and environmental samples. A sample may include a specimen of synthetic origin.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “organized cell cluster” refers to a plurality of cells (e.g., of the same or different cell types) that grow together and form a functional unit. Examples include, but are not limited to, ovarian follicles, matrix-directed cardioprogenitor cells, embryoid bodies, and primary cell co-cultures.

As used herein, the term “oocyte maturation” refers to biochemical events that prepare an oocyte for fertilization. Such processes may include but are not limited to the completion of meiosis II. The term “oocyte nuclear maturation” specifically refers to such completion of meiosis II. The term “oocyte cytoplasmic maturation” specifically refers to cytoplasmic events that occur to instill upon the oocyte a capacity to complete nuclear maturation, insemination, and/or early embryogenesis. Oocyte cytoplasmic maturation events may include but are not limited to accumulation of mRNA, proteins, substrates, and nutrients that are required to achieve the oocyte developmental competence that fosters embryonic developmental competence.

As used herein, the term “blastocyst” refers to a thin-walled hollow structure in early embryonic development that includes a cluster of cells called the inner cell mass from which the embryo arises.

As used herein, the term “follicle” or “ovarian follicle” refers to spherical aggregations of cells found in the ovary. They contain a single oocyte, which develops inside the follicle. Ovarian follicles comprise a number of different cell types surrounding the oocyte (e.g., granulosa cells and the follicular basement membrane or basal lamina).

As used herein, the term “cumulus cell” refers to a cell in the developing ovarian follicles which is in direct or close proximity to an oocyte. In some embodiments, the cumulus or cumulus cell refers to cells of the membrana granulosa that are collected into a mass which projects into the cavity of the follicle. This cluster of cells is released with the embedded oocyte during ovulation or following oocyte maturation.

As used herein, the term “medium” or “fluid medium” refers to any fluid within a system. In some embodiments, the medium or fluid medium is compatible with cell culture (e.g., supports cell viability; supports cell growth; supports cell development; does not cause toxicity or lethality to a cell).

### DETAILED DESCRIPTION OF THE INVENTION

The present invention relates to matrices (e.g., fibrin-alginate matrices; fibrin-alginate-matrigel matrices) for culture of cells, organs (e.g., ovary or fragment thereof), cells and cell aggregates (e.g., ovarian follicles, embryoid bodies), and tissues. In some embodiments, protease inhibitors (e.g., aprotinin) are used to prevent the degradation of fibrin.

The ovarian follicle consists of an oocyte surrounded by layers of granulosa cells, a basement membrane composed of ECM, and an outer layer of theca cells. As follicles develop, the somatic cells surrounding the oocyte proliferate and differentiate, and the oocyte grows in preparation for ovulation and fertilization. Communication between the multiple cellular compartments is essential for follicle development and oocyte maturation; thus, hydrogels have been employed for culture of ovarian follicles to support and maintain the normal follicular architecture (Kreeger et al. (2005) Biol. Reprod. 73:942-950; Pangas et al. (2003) Tissue Eng. 9:1013-1021; Kreeger et al. (2006) Biomaterials 27:714-723; West et al. (2007) Semin. Reprod. Med. 25:287-299; Xu et al. (2006) Biol. Reprod. 75:916-923; West-Farrell (2009) Biol. Reprod. 80:432-439; Xu et al. (2009) Biotechnol. Bioeng. 103:378-386; each herein incorporated by reference in its entirety. Mouse follicles encapsulated and cultured in alginate hydrogels grow, produce fluid-filled antral cavities, and meiotically competent oocytes, which yield multiple live births of healthy mouse pups. However, better methods are needed to to increase the number and quality of oocytes. While the present invention is not limited to any particular mechanism, and an understanding of the mechanism is not necessary to practice the present invention, it is contemplated that the alginate hydrogels, which are not degradable, become more rigid in the region adjacent to the follicle as the follicle increases in size. Soft hydrogels provide a more permissive environment relative to more rigid hydrogels (West et al. (2007) Biomaterials 28:4439-4448; Xu et al. (2006) Biol. Reprod. 75:916-923; West-Farrell et al. (2009) Biol. Reprod. 80:432-439; each herein incorporated by reference in its entirety).

During experiments conducted during the course of development of embodiments of the present invention, to facilitate development of methods for in vitro ovarian follicle culture, properties of the semi-degradable fibrin-alginate interpenetrating network (FA-IPN) were investigated to determine parameters resulting in dynamic cell-responsive mechanical properties. These cell-responsive mechanical properties were applied to the in vitro growth of ovarian follicles. The mechanical properties and polymerization rate of the gels and the fiber structure were analyzed by electron microscopy. Animals models described herein demonstrated the suitability of the matrices described for the culture and maturation of oocytes from multiple organisms.

In experiments conducted during the course of developing some embodiments of the present invention, an interpenetrating network of natural biomaterials that have dynamic cell responsive mechanical properties was created. Such networks provided a culture environment that supported tissue growth.

### I. Matrices

Embodiments of the present invention provide matrices for the culture of cells, organs and tissues. In some embodiments, matrices are interpenetrating networks (IPN). IPNs are a combination of polymers in network form, where at least one polymer is synthesized and/or crosslinked in the presence of the other, either simultaneously or sequentially (Sperling et al. (1996) Poymers for Adv. Technol. 7:197-208; herein incorporated by reference in its entirety). The chains of the individual polymers are completely entangled, and there may or may not be chemical bonds between the combined networks. This structure results in characteristics from each individual polymer being evident in overall IPN behavior (Rowe et al. (2006) Biomacromol. 7:2942-2948; herein incorporated by reference in its entirety).

In some embodiments, matrices are IPNs comprising fibrin and alginate. Fibrin forms a biomatrix with multiple ECM components and entrapped growth factors. Fibrinogen is a soluble 340 kDa protein that is polymerized into fibrin through the action of thrombin in the presence of calcium. Factor XIIIa, activated by thrombin, then crosslinks fibrin by linking a glutamine residue on the fibrinogen to a lysine on another. By comparison, alginate is a relatively inert scaffold and does not interact with integrins of mammalian cells (Werner et al. (2003) Physiol. Rev. 83:835-870; herein incorporated by reference in its entirety), yet forms a hydrogel under mild conditions, provides mechanical support to the tissue, and can be modified to present specific adhesion sequences (Augst et al. (2006) Macromol. Biosci. 6:623-633; Eiselt et al. (2000) Biomaterials 21:1921-1927; Kreeger et al. (2005) Biol. Reprod. 73:942-950; Pangas et al. (2003) Tissue Eng. 9:1013-1021; West et al. (2007) Biomaterials 28:4439-4448; Xu et al. (2007) Cancer Treat. Res. 138:75-82; each herein incorporated by reference in its entirety). Alginate, a naturally derived polysaccharide produced by brown algae, is used to support tissue growth.

The biologically degradable natural fibrin component of the IPN degrades as cells grow, expand, and secrete ECM component. The inert slow-degradable alginate component of the gel maintains the integrity of the matrix in order to provide the needed physical support. While the present invention is not limited to any particular mechanism, and an understanding of the mechanism is not necessary to practice the present invention, it is contemplated that the initial integrin concentration and the gel strength are likely determined by the fibrin component, yet alginate can be modified with ECM components to direct cell function after fibrin degradation.

In some embodiments, fibrin alginate matrices comprise thrombin and calcium as crosslinking agents. The present invention is not limited to a particular crosslinking agent. One of skill in the art recognizes that any number of suitable (e.g., bio-compatible) crosslinking agents are suitable for use in embodiments of the present invention. In some embodiments, concentrations of crosslinking agent are adjusted to limit the formation time of matrices (e.g., to 5-10 minutes) and still form strong fibers that resist degradation by cellular enzymes.

In some embodiments, matrices include protease inhibitors to delay the breakdown of matrix components (e.g., fibrin). Examples of protease inhibitors include but are not limited to aprotinin, 4-(2-aminoethyl)benzenesulfonyl fluoride (AEBSF), amastatin-HCl, alpha1-antichymotrypsin, antithrombin III, alpha1-antitrypsin, 4-aminophenylmethane sulfonyl-fluoride (APMSF), arphamenine A, arphamenine B, E-64, bestatin, CA-074, CA-074-Me, calpain inhibitor I, calpain inhibitor II, cathepsin inhibitor, chymostatin, diisopropylfluorophosphate (DFP), dipeptidylpeptidase IV inhibitor, diprotin A, E-64c, E-64d, E-64, ebelactone A, ebelactone B, EGTA, elastatinal, foroxymithine, hirudin, leuhistin, leupeptin, alpha2-macroglobulin, phenylmethylsulfonyl fluoride (PMSF), pepstatin A, phebestin, 1,10-phenanthroline, phosphoramidon, chymostatin, benzamidine HCl, antipain, epsilon-aminocaproic acid, N-ethylmaleimide, trypsin inhibitor, 1-chloro-3-tosylamido-7-amino-2-heptanone (TLCK), 1-chloro-3-tosylamido-4-phenyl-2-butanone (TPCK), trypsin inhibitor, and sodium EDTA. In some embodiments, inhibitors are neutralized or removed to increase the rate of growth of follicles and promote maturation of oocytes.

In some embodiments, fibrin alginate matrices are generated from TISSEEL fibrin sealant (Baxter Healthcare, BioScience Division, Westlake Village, Calif.) and alginate solutions, for example, as described below. One skilled in the art recognizes that additional methods of generating matrices are suitable for use in embodiments of the present invention.

Embodiments of the present invention provide kits and systems comprising one of the matrices described herein (e.g., fibrin-alginate matrix) and additional components necessary, sufficient, or useful in the growth and maturation of organized cell clusters (e.g., ovarian follicles). In some embodiments, the present invention provides systems comprise a matrix and one or more organized cell clusters, wherein said organized cell clusters interact with the matrix (e.g., are encapsulated in the matrix).

### II. Applications

As described above, embodiments of the present invention provide fibrin-alginate matrices for the culture and maturation of a variety of cell types. Exemplary uses of the matrices are described herein. The following examples are for illustrative purposes only. One skilled in the art understands that the matrices described herein find use in a variety of additional applications.

A. Oocyte Maturation

In some embodiments, the fibrin-alginate matrices described herein find use in the growth and maturation of ovarian follicles. Follicle development is regulated by many endocrine and paracrine factors, as well as the ECM of the follicle (Kreeger et al. (2005) Biol. Reprod. 73:942-950; Kreeger et al. (2006) Biomaterials 27:714-723; each herein incorporated by reference in its entirety). Antrum formation and steroidogenesis are two aspects of this developmental process and are influenced by the matrix. Mechanical properties of the matrix are a significant regulator of follicle development. A model for mechanical regulation of ovarian function suggested that immature follicles reside in the “less permissive” region of the ovary—the cortex (West et al. (2007) Biomaterials 28:4439-4448; herein incorporated by reference in its entirety). As immature follicles develop, they migrate to the medulla of the ovary, which is proposed to have a “more permissive” biomechanical environment. While the present invention is not limited to any particular mechanism, and an understanding of the mechanism is not necessary to practice the present invention, it is contemplated that small two-layered follicles cultured in a mechanically dynamic environment mimic this in vivo environment and increase the rate of oocyte maturation.

Accordingly, in some embodiments, the fibrin-alginate matrices described herein find use in the maturation and development of ovarian follicles. The matrices described herein are suitable for encapsulation of immature follicles. In some embodiments, encapsulated follicles develop and generate mature oocytes.

In some embodiments, oocytes matured using methods of embodiments of the present invention find use in in vitro fertilization. Experiments conducted during the course of development of embodiments of the present invention demonstrated that oocytes matured using the fibrin-alginate matrices described herein are suitable for use in in vitro fertilization and embryonic development.

The present invention is not limited to use with follicles from a particular animal. The methods of embodiments of the present invention find use with human ovarian follicles as well as follicles obtained from other animals (e.g., livestock, companion animals, etc.).

B. Cell Aggregates

In some embodiments, fibrin-alginate matrices find use in the growth of cell aggregates or clusters in which cell-cell contacts can be retained, yet the aggregate can degrade a matrix component to create space for expansion of the aggregate. Examples include, but are not limited to, matrix-directed cardioprogenitor cells (Kraehenbuehl et al. (2008) Biomaterials 29:2757-2766; herein incorporated by reference in its entirety), embryoid bodies (EBs) and primary cell co-cultures (Hoben et al. (2009) Stem Cells Dev. 18:283-292; herein incorporated by reference in its entirety).

### EXAMPLES

The following examples are provided in order to demonstrate and further illustrate certain preferred embodiments and aspects of the present invention and are not to be construed as limiting the scope thereof.

### Example 1

### Interpenetrating Fibrin-Alginate Semi-Degradable Matrices for In Vitro Ovarian Follicle Development

### A. Materials and Methods

Animals and Materials

Two-layered secondary follicles were mechanically isolated from 12-day-old female F1 hybrids (C57BL/6j×CBA/Ca). Animals were purchased (Harlan, Indianapolis, Ind.), housed in a temperature and light controlled environment (12 L: 12 D) and provided with food and water ad libidum. Animals were fed Teklad Global irradiated 2919 chow, which does not contain soybean or alfalfa meal and therefore contains minimal phytoestrogens. Unless otherwise noted, all chemicals were purchased from Sigma-Aldrich (St. Louis, Mo.), stains and antibodies from MolecularProbes (Eugene, Oreg.), and media formulations from Invitrogen (Carlsbad, Calif.). Sodium alginate (55-65% guluronic acid) was provided by FMC BioPolymers (Philadelphia, Pa.) and Tisseel® fibril sealant product (Baxter Healthcare, BioScience Division, Westlake Village, Calif.), was used for fibrin gels preparation.

FA-IPN Preparation

The fibrinogen-containing component of Tisseel ® was reconstituted in aprotinin (3000 KIU/mL) solution and the thrombin component was reconstituted in 40 mM CaCl₂, according to the manufacturer's instructions. Both solutions were diluted to the appropriate concentrations by diluting the fibrinogen containing component in Tris-buffered saline solution (TBS) and thrombin in 40 mM CaCl₂ in TBS. Alginate aliquots were prepared as previously described Pangas et al. (2003) Tissue Eng. 9:1013-1021; Kreeger et al. (2006) Biomaterials 27:714-723; each herein incorporated by reference in its entirety) and diluted to 0.5% w/v. IPNs were prepared by mixing fibrinogen solution (50 mg/mL) with alginate solution 0.5% in 1:1 ratio, and then adding thrombin solutions of 5, 50 and 500 IU/mL to the mixture at 1:1 ratio. The fibrinogen-alginate mix and the thrombin solutions were filled with equal volumes in 1 mL syringes and injected using the Duploject System provided with the kit, while mixing in the needle. Thus, the final concentrations in the gels of fibrinogen and alginate were 12.5 mg/mL and 0.125%, respectively.

FA-IPN Characterization

FA-IPNs shear elastic modulus was measured at 25° C. using a Paar Physica MCR Rheometer (Anton Paar, Graz, Austria) using a parallel plate geometry (diameter of 25 mm, gap of 0.5 mm) and Paar Physica US200 software. For each of the 3 different concentration of thrombin the FA gels were extruded on the lower plate of the rheometer. The upper plate was immediately lowered, and the gels crosslinked between the plates for 10 min. The limits of linear viscoelasticity were determined in strain sweep experiment with strain range from 0.1% to 100% at a constant angular frequency of 10 rad/s. Storage and loss moduli were determined in amplitude sweep experiments at constant strain of 0.5% and angular frequency range from 100 to 0.1 rad/s. The gels were formed and let crosslink for 10 min before the experiment started. For the gelation rate measurement the gels were extruded on the lower plate and the experiment was started immediately. The gelation profiles for 3 different concentrations of thrombin were performed at 10 rad/s angular frequency and 0.5% strain for 30 min. Fibrin gel prepared with 25 mg/mL fibrinogen and 50 IU/mL thrombin concentrations, and alginate gels at concentrations of 0.125% and 0.5% were used as a control conditions.

Follicle Isolation, Encapsulation and Culture

Two-layered secondary follicles (100-130 μm, type 4) were mechanically isolated as described (Pangas et al. (2003) Tissue Eng. 9:1013-1021; West et al. (2007) 28:4439-4448; Kreeger et al. (2006) Biomaterials 27:714-723; Xu et al. (2006) Biol. Reprod. 75:916-923; each herein incorporated by reference in its entirety) and encapsulated in FA-IPNs or fibrin gels. Fibrinogen-alginate solutions (7.5 μL, 25 mg/mL fibrinogen, 0.25% alginate) were pipetted on alcohol wiped glass slide with 3 mm spacers and covered with parafilm and individual follicles were transferred into the droplets using a pipette. Thrombin solutions (7.5 μL of 5, 50 or 500 IU/mL) were pipetted on top of each droplet with the follicle. The droplets were covered with the second glass slide covered with alcohol wiped parafilm and transferred to the 37° C. incubator for 5 min. The beads with the follicles were washed in maintenance media (αMEM, 1 mg/mL bovine serum albumin and penicillin-streptomycin) and transferred to 96-well plates with 150 μl of culture media (αMEM, 3 mg/mL bovine serum albumin (MP Biomedicals, Inc., Solon, Ohio), 10 mIU/mL rFSH, 1 mg/mL bovine fetuin, 5 μg/mL insulin, 5 μg/mL transferrin and 5 ng/mL selenium). As a control, follicles were encapsulated in fibrin only beads or fibrin gels formed in culture plate inserts (0.4 μm, 12 mm diameter, Millipore, Billerica, Mass.). Beads made of fibrin only were formed in the same manner as described previously for FA. For cultures in the insert, fibrinogen solution (25 mg/mt, 50 μL) was pipetted into the insert, and 5-7 follicles were added with thrombin solution (50 IU/mL, 50 μL) pipetted over the follicles. The gels were allowed to form for 10 min. The inserts were transferred to 24 well plates and covered with culture media. Throughout the isolation, encapsulation and plating, follicles were maintained at 37° C. and pH 7.

Encapsulated follicles were cultured at 37° C. in 5% CO₂ for 12 days. Every other day, half of the media (75 μL) was exchanged and stored at −80° C. for steroid assay. Follicle survival and diameter were assessed using an inverted Leica DM IRB microscope with transmitted light (Leica, Bannockburn). The diameter of follicles containing oocytes was measured in duplicate from the outer layer of theca cells using ImageJ 1.33U (NIH) and based on a calibrated ocular micrometer.

Oocyte Meiotic Competence

Oocyte meiotic competence was assessed by maturation after 12 days of culture. Follicles were removed from the beads by 10 min incubation of the beads in a 10 IU/mL solution of alginate lyase, which enzymatically degrades the alginate, in prewarmed L-15 media. Antral follicles were transferred to αMEM containing 10% FBS, 5 ng/mL epidermal growth factor and 1.5 IU/mL human chorionic gonadotropin and were matured at 37° C. in 5% CO₂ for 14-16 h. Oocytes were denuded then from the surrounding cumulus cells by treating with 0.3% hyaluronidase. Oocyte state was assessed from the light microscopy images, and characterized as follows: germinal vesicle breakdown (GVBD) if the germinal vesicle was not present, GV if there was an intact germinal vesicle, metaphase II (MII) if a polar body was present in the perivitelline space and degenerated (DG) if the oocyte was fragmented or shrunken.

Follicle Fixation and H&E Staining

Follicles were fixed inside FA-IPN bead. The fixation was performed at 4° C. overnight in 4% paraformaldehyde (PFA), dehydrated in ascending concentrations of ethanol (50-100%), and embedded in paraffin by an automated tissue processor (Leica, Manheim, Germany). Serial 5 μm sections were cut and stained with hematoxylin and eosin (PO1 Ovarian Histology Core facility).

Oocyte Preparation for Confocal Microscopy and Imaging

Matured oocytes were fixed in 4% PFA for 2 hours at room temperature and stored in wash solution containing 0.2% azide, 2% normal goat serum, 1% BSA, 0.1 M glycine, and 0.1% Triton X-100 at 4° C. until further processing. Oocytes were immunolabeled to ascertain maturation state, centrosome, spindle and polar body position and shape. A total of 15 oocytes per gel condition were incubated in primary antibody (α/β tubulin cocktail 1:100; mouse; Sigma) in 4° C. overnight with gentle agitation, followed by three 10-min washes in wash buffer, followed by 1-hour incubation of secondary antibody (Alexa 488 goat anti-mouse IgG 1:500; Molecular Probes) with rhodamine-phalloidin (1:500; Molecular Probes) at room temperature. Oocytes were mounted in 2 μL of a 50% glycerol/PBS solution containing 1 μg/mL Hoechst 33258 to label chromatin. Samples were analyzed on an inverted Nikon Cl Si Multispectral Laser Scanning Confocal Microscope (Nikon Instruments, NY) equipped with a 100-W mercury arc lamp and were imaged using 40× and 63× objectives. A triple band pass dichroic and automated excitation filter selection specific for fluorescein (Alexa 488), rhodamine (Alexa 568) and bisbenzimides (Hoechst 33258) permitted the collection of in-frame images and z axis data sets at 0.5 μm intervals.

Steroid Assays

Androstendione, 17β-estradiol and progesterone were measured in collected media from 12-day individual follicle culture using commercially available radioimmunoassay kits (androstendione and 17β-estradiol, Diagnostic Systems Laboratories, Inc., Webster, Tex.; progesterone, Diagnostic Products Corporation, Los Angeles, Calif.). The media from the same condition and time point were pooled, and each condition was tested in triplicate. The sensitivities for the androstendione, estradiol and progesterone assays are 0.1, 10 and 0.1 ng/mL, respectively.

Statistical Analysis

Statistical calculations were performed using JMP 4.0.4 software (SAS Institute, Cary, N.C.). Statistical significance for follicle size measurements and steroid levels was analyzed using a two-way ANOVA with repeated measures, or one-way ANOVA followed by Tukey-HSD for single time points. Values of p<0.05 were considered significant.

### B. Results

FA-IPN Characterization

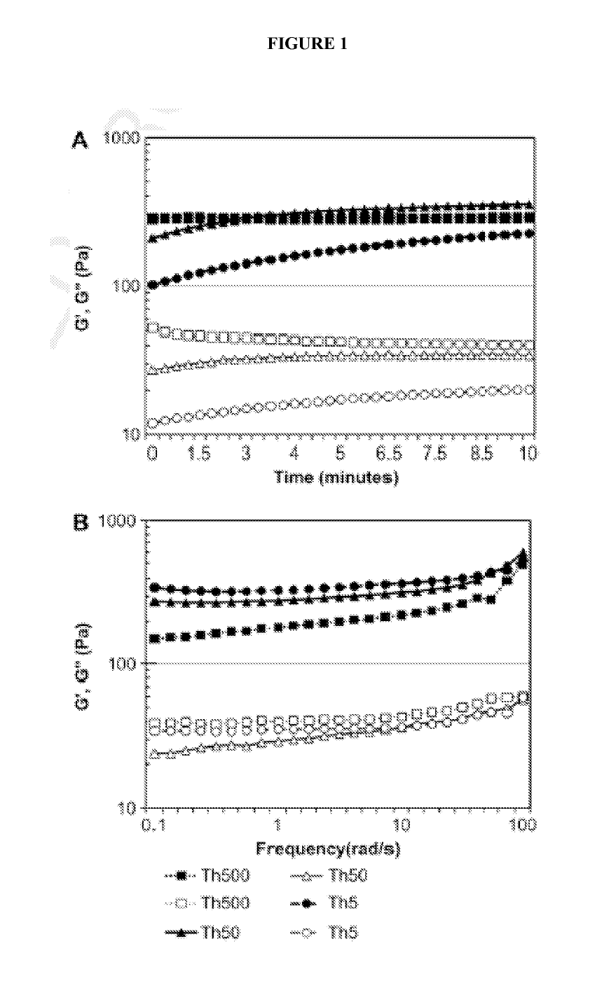

Fibrin-alginate IPNs were formed with three concentrations of thrombin. Both components of the IPN, the fibrinogen and the alginate, started to crosslink immediately as they were mixed with the crosslinker, thrombin and Ca²⁺, and a longer duration of crosslinking resulted in stronger gels. While the present invention is not limited to any particular mechanism, and an understanding of the mechanism is not necessary to practice the present invention, it is contemplated that as crosslinking was initiated, the storage modulus (G′) of the IPN increased with thrombin content (FIG. 1A). The storage modulus was 100 Pa, 220 Pa and 280 Pa for thrombin concentrations of 5 IU/mL, 50 IU/mL and 500 IU/mL, respectively. With increasing time, the dependence of the storage modulus on the thrombin content decreased. After the 30 min, the moduli were equal to 350, 370 and 300 Pa for 5, 50 and 500 IU/mL thrombin, respectively. The low thrombin concentration resulted in a slower rate of crosslinking, yet produced a higher storage modulus after 30 min. A 100 fold higher thrombin concentration formed a gel immediately with no increase in storage modulus over time. In amplitude sweep experiments, IPNs were crosslinked for 10 min and then tested at constant strain of 0.5% with decreasing angular frequency from 100 to 0.1 sec⁻¹ (FIG. 1B). Higher thrombin concentration resulted in IPNs with lower storage modulus: 235 Pa for 500 IU/mL thrombin, 300 Pa for 50 IU/mL thrombin, and 370 Pa for 5 IU/mL thrombin. The similar amplitude sweep experiment was used to determine the storage modulus for fibrin gel (25 mg/mL fibrinogen; 50 IU/mL thrombin), alginate 0.5% and 0.125% (Table 1).

TABLE 1
Hydrogel characterization: storage modulus data represented as
average from three or more independent measurements.

| | Storage modulus (G′) at 0.5% strain, |
|---|---|
| Gel | 10 sec⁻¹ angular frequency |
| ALG/Fibrin Th 5 | 370 Pa |
| ALG/Fibrin Th 50 | 300 Pa |
| ALG/Fibrin Th 500 | 235 Pa |
| Fibrin | 300 Pa |
| ALG 0.5% | 300 Pa |
| ALG 0.125% | 42 Pa |

G′ = storage modulus,
Pa = Pascal

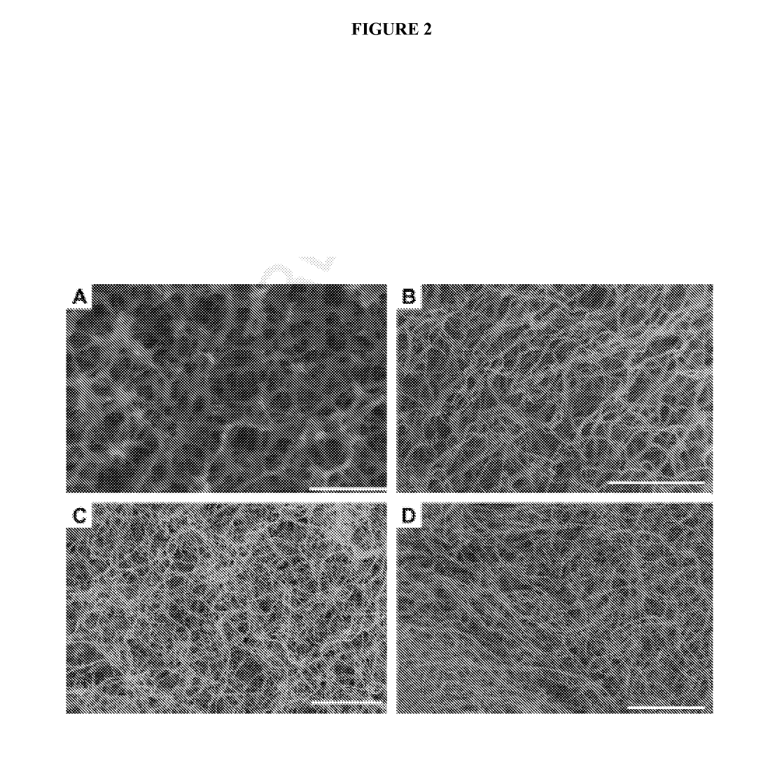

The architecture of the FA-IPN produced at a range of thrombin concentrations was investigated. For the three thrombin concentrations tested, IPN gels had a network of fibers, with the fiber diameter ranging from 15-120 nm (FIG. 2). IPNs prepared with 500 IU/mL thrombin resulted in more visually dense and compact matrix with thinner fibers compared to IPN formed at lower thrombin concentrations.

Two-Layered Secondary Follicle Encapsulation and Growth

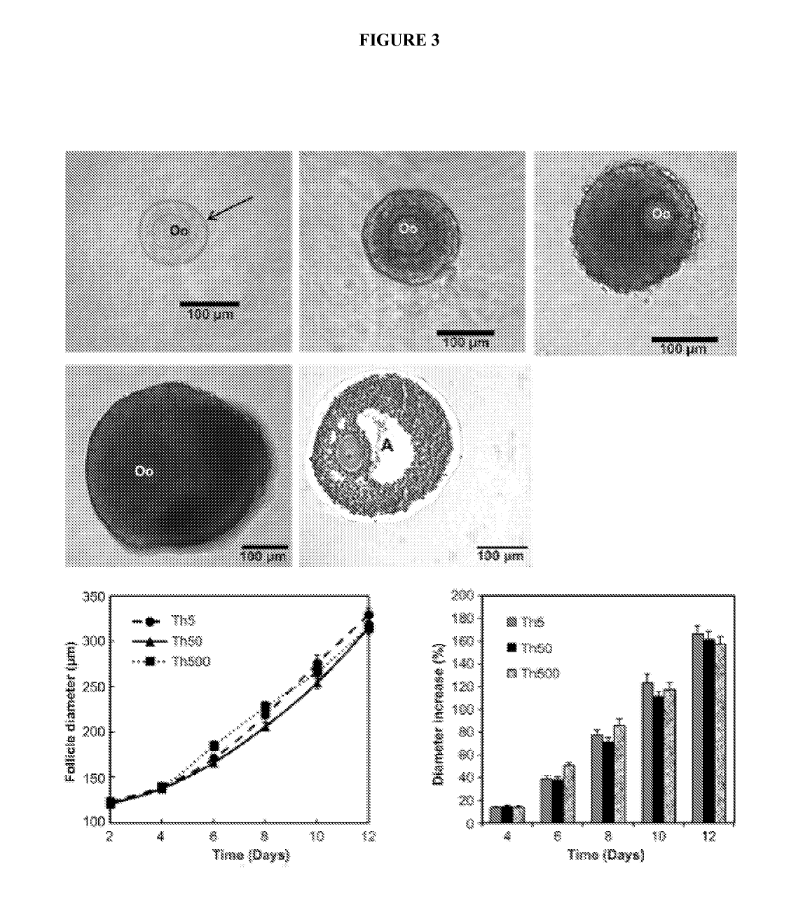

Follicle development was investigated in FA-IPNs prepared with increasing (5, 50 and 500 IU/mL) concentrations of thrombin. A minimum of 80 follicles were encapsulated and cultured for 12 days. Follicles maintained their spherical 3D structure while growing from small follicle with an oocyte surrounded by two layers of granulosa cells on day 2 of the culture (FIG. 3A), with growing number of granulosa cells layers on days 4 and 8 (FIGS. 3B and C). A fluid filled antrum cavity was observed on day 12 (FIG. 3D), which is consistent with in vivo morphology. Antral follicle fixed on day 12 from the culture, sectioned and stained with H&E had a spherical shape with a central antrum and oocyte surrounded by cumulus cells (FIG. 3E). The survival rate of the follicles was the same for all the conditions (77-81%, Table 2) and was similar to the previously reported rates in alginate cultured follicles (Xu et al. (Biol. Reprod. (2006) 75:916-923; herein incorporated by reference in its entirety). In all groups, follicles increased in their diameter from 120 μm on day 2 to 330 μm on day 12, which is an increase of 160% (FIGS. 3F and G).

Fibrin Degradation

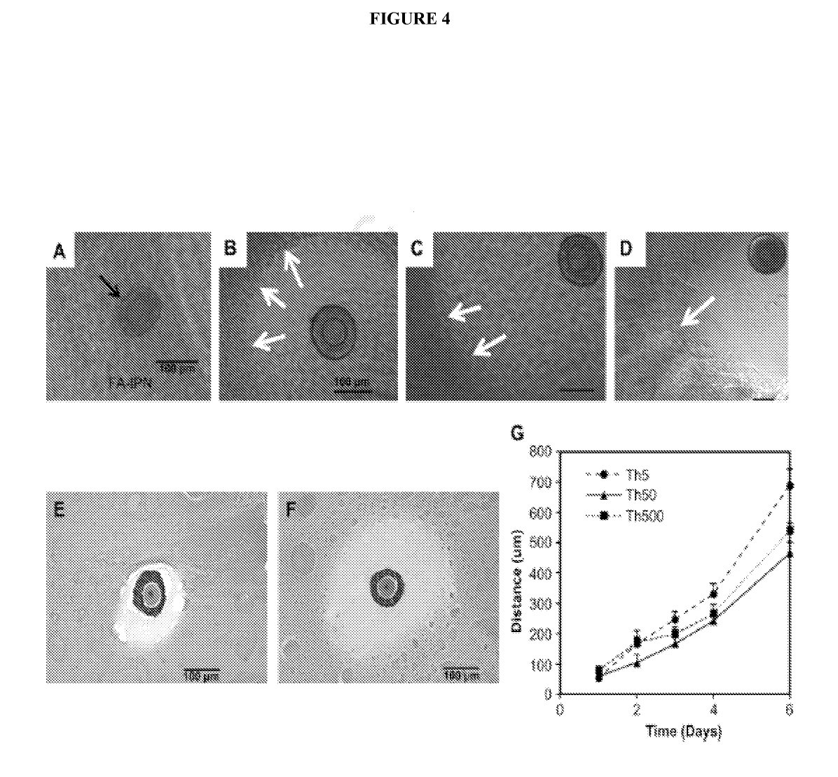

The fibrin component of the IPN gradually disappeared from the hydrogel, with the initial clearance occurring adjacent to the follicle and then progressing toward the edge of the hydrogel bead (FIG. 4). The follicles were initially difficult to image due to light diffraction be the FA-IPN, which made the hydrogel appear cloudy (FIG. 4A). On the second day of a culture, the region immediately adjacent to the follicle became clear, consistent with the degradation of the fibrin component of the IPN. The non-degradable alginate component remained and supported the 3D architecture of the growing follicle (FIG. 4B-D). The distance from the edge of the encapsulated follicle to the edge of the degradation front was measured. Fibrin in 5 IU/mL thrombin degraded fastest relative to the other two conditions, reaching a distance of 690 μm in 6 days of culture. For 50 IU/mL and 500 IU/mL thrombin the degradation occurred at slower rates, reaching 470 μm and 540 μm, respectively (FIG. 4E). Follicles cultured for 2 and 4 days (FIGS. 4F and 4G) had degradation around the follicle, with the fibrin-alginate matrix remaining intact beyond the degradation front.

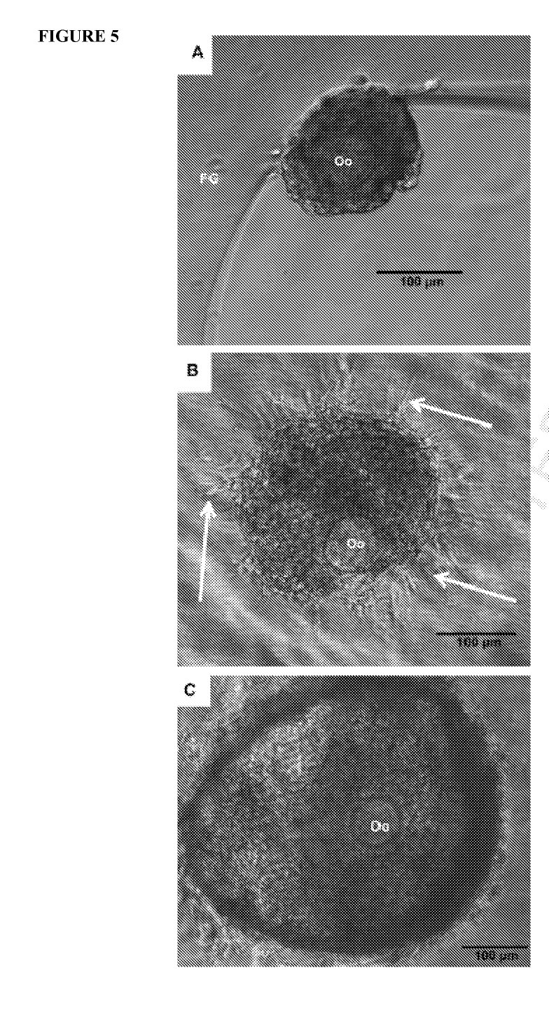

Control studies were performed with fibrin hydrogels without alginate to investigate whether fibrin alone would be sufficient to promote follicle growth. Studies using alginate alone have been previously reported (West et al. (2007) Biomaterials 28:4439-4448; Xu et al. (2006) Biol. Reprod. 75:916-923; each herein incorporated by reference in its entirety). Follicles were cultured in fibrin gels using two different methods of encapsulation: beads and culture plate inserts, or transwells. Follicles cultured in fibrin beads degraded the surrounding fibrin, and by day 6, the follicles either lost their spherical shape or were no longer within the fibrin (FIG. 5A). While the present invention is not limited to any particular mechanism, and an understanding of the mechanism is not necessary to practice the present invention, it is contemplated that upon degradation of the fibrin, the mechanical support of the hydrogel was lost leading to changes in shape of the follicle. At day 6, the follicles stopped growing and were no longer viable. Due to this loss of support, follicles were subsequently cultured within the transwell inserts, which prevented loss of the follicle from the bead. In this condition the granulosa cells spread and migrated into the surrounding fibrin and follicles lost their typical spherical shape (FIG. 5B). At the end of a culture in inserts, follicles reached 600 μm in their diameter, but their shape was similar to 2D cultured follicles (i.e., flattened with poorly connected granulosa cells) (FIG. 5C).

Functional Analysis

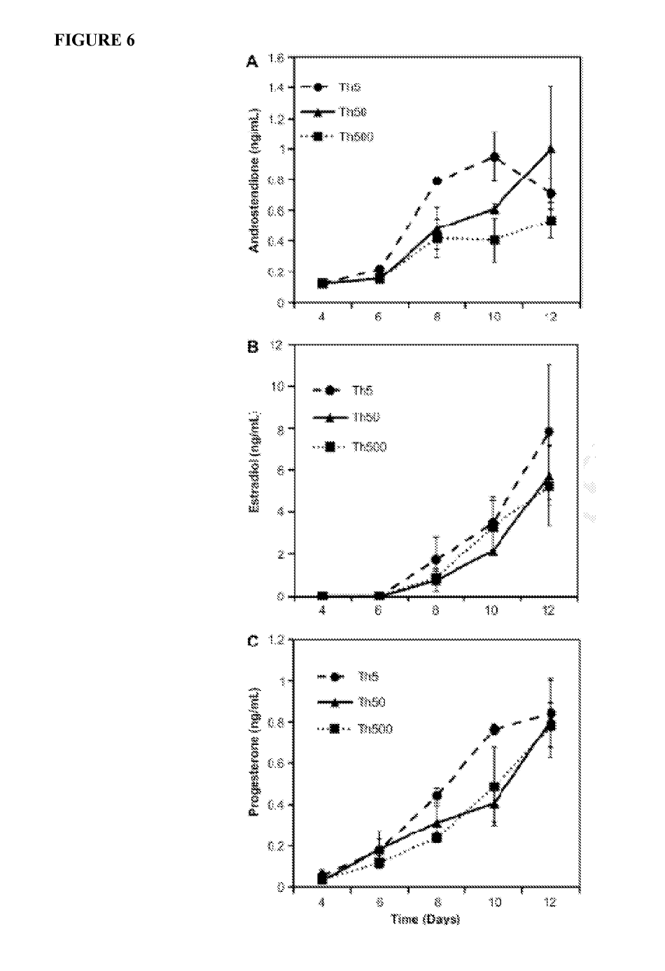

The functional development of the follicles within the FA-IPN was analyzed. Steroid production by the follicle was quantified. Androstenedione (A) levels in all three conditions increased from baseline on day 6 and was significantly greater in 5 IU/mL thrombin (0.8 and 1 ng/mL on days 8 and 10) than in 50 IU/mL thrombin and 5 IU/mL thrombin (FIG. 6A). However, on the last day of culture no significant difference was observed between conditions. Progesterone (P) levels, similar to androstenedione, increased on day 6 with greater levels in 5 IU/mL thrombin (0.8 ng/mL) on day 10 compared to other two conditions (0.8 ng/mL and 0.5 ng/mL, respectively). On day 12 of culture, all groups had similar progesterone concentration of 0.8 ng/mL (FIG. 6B). Estradiol (E) levels increased on day 8 and reached maximum concentration on day 12 (6-8 ng/mL) with no significant difference between the conditions (FIG. 6C). Appropriate quantities of steroid biosynthesis are reflected in the ratio of secreted estradiol, androstenedione and progesterone (Table 2). In all thrombin conditions described herein, the ratio A/E and P/E was 0.1-0.2, indicating that follicle development was supported in FA-IPNs.

TABLE 2
Ratios of estradiol concentration to androstenedione and progesterone
concentrations at end of culture

| | E | A | P |
|---|---|---|---|
| Th5 | 1 | 0.09 | 0.11 |
| Th50 | 1 | 0.17 | 0.14 |
| Th500 | 1 | 0.10 | 0.15 |

E = estradiol,
A = androstendione,
P = progesterone.

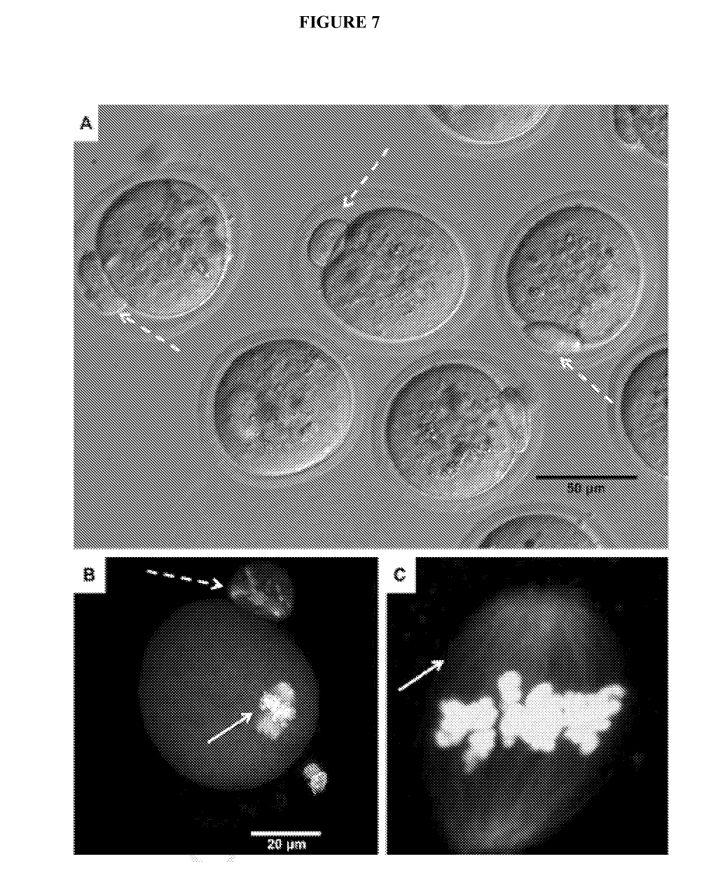

The quality of oocytes obtained from follicles cultured within FA-IPNs was subsequently measured by their ability to resume meiosis. Oocytes from follicles cultured in all thrombin conditions demonstrated high rate (75-82%, Table 3) of Metaphase II (MII) stage and polar body extrusion (FIG. 7A). This rate of MII stage oocytes obtained from follicles cultured in FA-IPNs was significantly higher than previously reported for 0.25% alginate system (67.2%). The percentage of GV oocytes was similar for all conditions, but in 500 IU/mL thrombin, a greater percentage of degenerated oocytes was observed (16% in 500 IU/mL thrombin versus 6% in 50 IU/mL thrombin). For the analysis of spindle structure and chromosome positioning, MII stage oocytes obtained from cultured follicles were stained with a fluorescent antibody to β-tubulin and DAPI, and imaged with confocal microscope (FIGS. 7B and 7C). Oocytes that extruded the first polar body exhibited a normal MII configuration, with microtubules organized into a bipolar spindle and the chromosomes tightly aligned on the spindle equator.

TABLE 3
Survival rates, follicle size measurement and meiotic competence
rates from two-layered secondary follicles cultured in
FA-IPNs in vitro.

| | Survival | Day 12 | MII | GVBD | GV | DG |
|---|---|---|---|---|---|---|
| Condition | (%)^{a} | diameter | (%) | (%) | (%) | (%) |
| ALG 0.25%^{b} | 78 | 326.5 | 67^{c} | 88^{c} | 4^{c} | 9^{c} |
| Th 5 | 77 | 329 | 76^{d} | 72^{d} | 8^{c} | 11^{c} |
| Th 50 | 81.3 | 314.6 | 82^{e} | 88^{e} | 6^{c} | 6^{d} |
| Th 500 | 78.1 | 315.4 | 75^{d} | 78^{d} | 6^{c} | 16^{e} |

^{a}Values are the average of multiple follicles from five independent cultures
^{b}Data from Xu et al. (2006) Biol. Reprod. 75: 916-923
^{c-e}Different superscripts within each column indicate statistically significant differences (p < 0.05).

### Example 2

### In Vitro Cultured Preantral Follicles from Luteal-Phase Baboon Ovaries Produce Oocytes Competent for In Vitro Maturation and Fertilization

### A. Materials and Methods

### Ovary Collection

Ovaries were obtained from 6 adult cycling baboons during the luteal phase, days 7-10 post-ovulation (PO) (Table 4). Ovulation was detected by measuring peripheral serum levels of estradiol, beginning 7 days after the first day of menses. The day of the estradiol surge was designated Day −1, with Day 0 as the day of the ovulatory LH surge and Day 1 as the day of ovulation. Luteal-phase ovaries were confirmed by presence of a corpus luteum (CL). Ovaries were transported to the laboratory at room temperature and less than 1 hour after retrieval.

### Cumulus-Oocyte-Complex (COC) Isolation and Classification

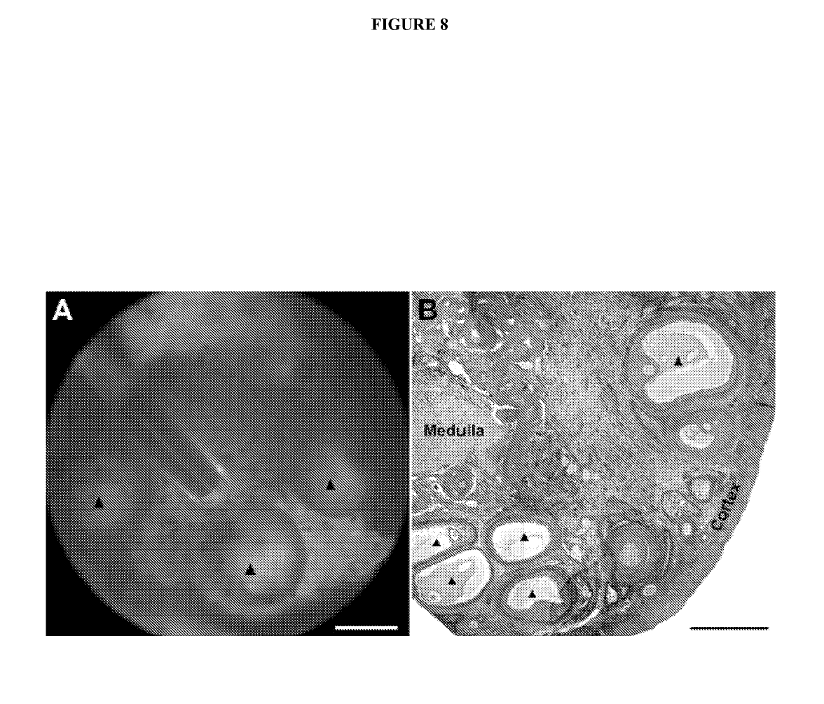

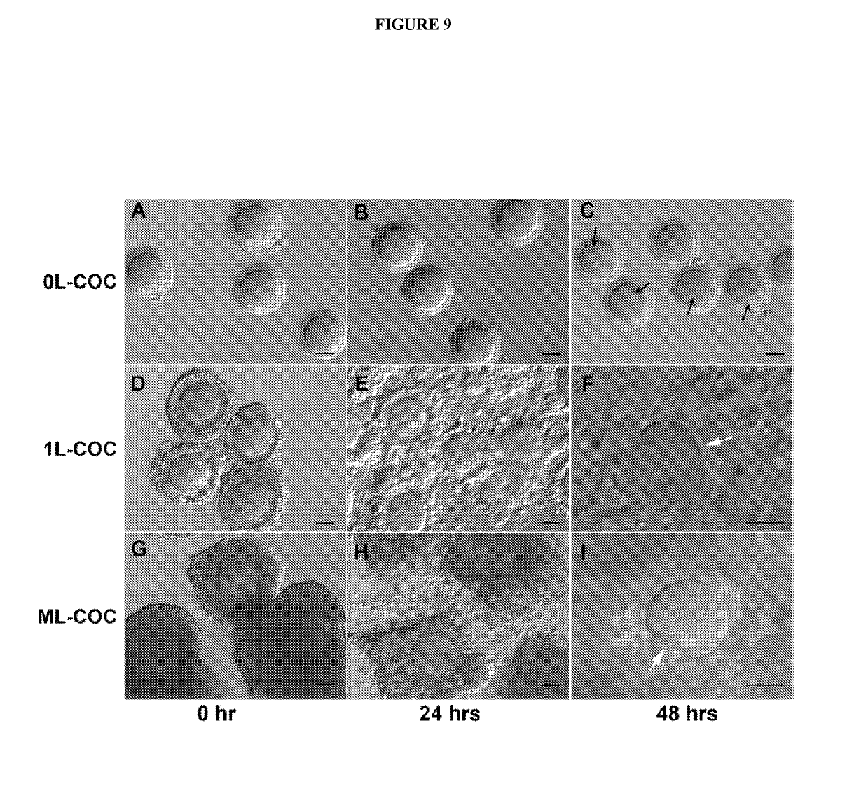

Ovaries were cut into quarters with a scalpel, and the medulla was separated from the cortex using curved scissors in MOPS-HTF medium (Cooper-Surgical, Trumbull, Conn.). COCs and preantral follicles were collected using methods described previously, with modifications. Small antral follicles (FIG. 8) located on the border between the cortex and medulla were punctured with a 25-gauge needle and gently squeezed to release the COCs. Using a dissecting stereomicroscope, COCs were classified to 3 groups: 0L-COC, incomplete layer of cumulus cells (FIG. 9A); 1L-COC, 1-2 complete layers of cumulus cells (FIG. 9D); and ML-COC, at least 3 complete layers of compact cumulus cells (FIG. 9G). COC classification was performed by a single observer to ensure uniformity of COC types.

### Preantral Follicle Isolation

The ovarian cortex was cut into small pieces (approximately 1-2 mm³) and the tissue was enzymatically digested in αMEM (Invitrogen, Carlsbad, Calif.) containing 1% HSA (Irvine Scientific, Santa Ana, Calif.), 0.08 mg/ml Liberase Blendzyme 3 (Roche Diagnostics, Indianapolis, Ind.), and 0.2 mg/ml DNase (Worthington Biochemical, Lakewood, N.J.) for 1 hour in a shaker incubator at 37° C. and 5% CO₂. After rinsing the cortex twice with MOPS-HTF medium, follicles were mechanically isolated using a 25-gauge needle into MOPS-HTF medium. The follicles were transferred to maintenance media (αMEM, supplemented with 1% HSA, 100 IU/ml penicillin and 100 μg/ml streptomycin) and placed in an incubator at 37° C. and 5% CO₂. Only preantral follicles (class 1 and 2) that contained a clear, visible, centrally-located oocyte, healthy granulosa cells, and no signs of antrum formation were encapsulated and cultured.

### Matrix Preparation

Sodium alginate (55-65% guluronic acid) was provided by FMC BioPolymers (Philadelphia, Pa.), Tisseel™ was provided by Baxter Healthcare (BioScience Division, Westlake Village, Calif.), and Growth Factor Reduced BD Matrigel™ (GFR-Matrigel) was purchased from BD Bioscience (BD Cat 354234, Bedford, Mass.). All biomaterials were prepared as described previously. Briefly, sterile alginate aliquots were reconstituted to 0.5% (w/v) in 1× PBS. Fibrinogen was reconstituted to 50 mg/ml in aprotinin (3000 KIU/mL) solution, and the thrombin component was reconstituted to 50 IU/ml in 50 mM CaCl₂/140 mM NaCl, according to the kit instructions (Baxter Healthcare). GFR-Matrigel was thawed on ice before use.

### Follicle Encapsulation and Culture

In order to fully deactivate the activity of the enzymes used for follicle isolation, preantral follicles were first embedded in 25% GFR-Matrigel for 1 hour as follows. GFR-Matrigel was diluted 1:3 with cold αMEM and added to a V-bottom 96-well plate. After 10 minutes at room temperature, individual follicles were transferred into each well and the plate was incubated for 50 minutes. Follicles were then retrieved from the Matrigel using blunt tip forceps.

The FAM matrix was prepared by mixing 25 μl fibrinogen (50 mg/ml), 25 μl alginate solution (0.25%), 40 μl 1×PBS and 10 μl GFR-Matrigel. Five to ten follicles were transferred immediately into the FAM mixture with a minimal amount of media. Using a 10-μl pipette tip, individual follicles in 5 μl of the FAM mixture were pipetted into the 50 IU/ml thrombin solution for crosslinking for 5 minutes. Fresh FAM mixture was prepared every 30 minutes until the encapsulation was completed. The crosslinked FAM beads, each containing a single follicle, were rinsed in maintenance media and plated one per well in 96-well plates in 100 μl of basal culture media: αMEM, 3 mg/ml HSA, 1 mg/ml bovine fetuin (Sigma-Aldrich, St. Louis, Mo.), 5 μg/ml insulin, 5 μg/ml transferrin, and 5 ng/ml selenium. Throughout isolation, encapsulation, and plating, follicles were maintained at 37° C. and pH 7. Encapsulated follicles were cultured at 37° C. in 5% CO₂ up to 14 days. Every other day, half of the media (50 μl) was exchanged and stored at −80° C. for use in steroid assays.

### Follicle Culture in FSH-Containing Media

In the first experimental culture phase, follicles from 4 baboons were randomly separated into 3 groups and grown for 10 days in culture media supplemented with 0, 10, or 100 mIU/ml recombinant human FSH (NV Organon, Oss, The Netherlands). Follicles were then recovered and oocytes underwent IVM. In the second phase, follicles from 2 baboons were grown for 14 days in the absence of FSH, which was the culture condition identified in phase 1 that produced the highest rate of meiotically competent oocytes. Follicles were then recovered and the oocytes underwent IVM.

### In Vitro Maturation (IVM)

Oocyte maturation was carried out using an IVM kit (Cooper-Surgical) at 37° C. in 5% CO₂ for 46-48 hours. The vendor-supplied IVM media was supplemented with 100 mIU/ml FSH (NV Organon), 100 mIU/ml LH (Ares Serono, Randolph, Mass.), 1 IU/ml human chorionic gonadotropin (hCG) (Sigma), 10 ng/mL epidermal growth factor (EGF) (Sigma), and 5% (v/v) heat inactivated fetal bovine serum (FBS) (Invitrogen).

Ten to fifteen of each class of COCs from small antral follicles underwent IVM in an Organ Tissue Culture Dish (60×40 mm; Falcon/BD Biosciences, San Jose, Calif.) containing 1 ml maturation media covered with embryo-grade mineral oil.

In vitro cultured preantral follicles were first removed from the FAM matrix beads by incubation in a 10 IU/mL solution of alginate lyase in prewarmed MOPS-HTF medium for 10 minutes. The COCs were carefully separated from the surrounding follicle using 2 28-gauge insulin needles and individual COCs were transferred into a 15-μl droplet of IVM media covered with embryo-grade mineral oil. Oocytes were then denuded of cumulus cells with 0.3% hyaluronidase. Oocyte state was assessed using light microscopy, and characterized as follows: germinal vesicle breakdown (GVBD) if the germinal vesicle was not present; GV if there was an intact germinal vesicle; metaphase II (MII) if a polar body was present in the perivitelline space; and degenerated (DG) if the oocyte was fragmented or shrunken.

### Intracytoplasmic Sperm Injection (ICSI) and Embryo Culture

Mature oocytes were inseminated by ICSI with frozen-thawed baboon sperm. Fertilization was evaluated 16-18 hours after injection and was considered normal when two pronuclei were observed. Embryos were individually cultured for 5 days in a 20-μl drop of embryo culture media provided in the IVM kit (Cooper-Surgical) under mineral oil at 37° C. in 5% CO₂.

### Follicle and Oocyte Measurement

During the follicle culture period, photographs of each follicle were captured using a Leica DM IL light microscope (Leica, Wetzlar, Germany) equipped with phase objectives, a heated stage, a Spot Insight 2 Megapixel Color Mosaic camera, and Spot software (Spot Diagnostic Instruments, Sterling Heights, Mich.). Follicle diameters were later measured using ImageJ software (National Institutes of Health, USA) as previously described. Oocyte diameters, minus the zona pellucida, were measured on day 0, when the oocyte was enclosed in the follicle, and on day 14, when the COC had separated from the surrounding follicle.

### Tissue Sectioning and Staining

In vitro cultured follicles were fixed in 4% paraformaldehyde in 1×PBS overnight at 4° C. Follicles were dehydrated in ascending concentrations of ethanol (70%-100%), and embedded in paraffin using an automated tissue processor (Leica, Manheim, Germany). Serial 4-μm sections were cut and stained with hematoxylin and eosin.

### Immunostaining and Confocal Imaging

Oocytes were fixed and extracted in a microtubule-stabilizing buffer with 4% formaldehyde at 37° C. for at least 30 minutes. To visualize spindle and chromosome alignment, oocytes were incubated with mouse monoclonal anti-a-tubulin (1:200, Sigma) overnight at 4° C., followed by Alexa Fluor 488-conjugated rabbit-anti-mouse IgG (1:400, Molecular Probes, Eugene, Oreg.) for 1 hour at 25° C., and then were mounted in VectaShield with 1 μg/ml propidium iodide (PI, Vector Laboratories, Burlingame, Calif.). Images were obtained using a laser scanning confocal microscope (Leica TCS SP5×, Manheim, Germany) under a ×63 oil immersion objective. For each spindle, a complete Z-axis scan was collected at 0.5-μm intervals, and 3D projection was analyzed on Leica SP5 software.

### Hormone Assays

Androstenedione, 17β-estradiol, and progesterone were measured by hormone-specific electrochemoluminescent assay using a Roche Elecsys 2010 Analyzer (Roche, Indianapolis, Ind.). The interassay variations were 6.1% for 17β-estradiol and 5.4% for progesterone. The limits of sensitivity were 5 pg/ml for 17β-estradiol and 0.03 ng/ml for progesterone Inhibin A, inhibin B, and anti-Müllerian hormone (AMH) were measured using ELISA kits (DSL-10-28100, DSL-10-84100 and DSL-10-14400, Diagnostic Systems Laboratories, Webster, Tex.) following manufacturer instructions. The intra-assay variations were 8.7% for inhibin A, 3.2% for inhibin B and 3.8% for AMH. The limits of sensitivity for inhibin A, inhibin B, and AMH were 10 pg/ml, 10 pg/ml, and 20 pg/ml, respectively.

### Statistics

Maturation data were analyzed using one-way ANOVA, followed by a paired t-test. Spindle data analysis was carried out with pooled data using chi-squared analysis. P<0.05 was considered statistically significant. All statistical calculations were performed using the software GraphPad Prism version 4.0.

### B. Results

In Vitro Maturation of Oocytes from Small Antral Follicle COCs

To investigate the role of cumulus cells in promoting oocyte health, 371 COCs were collected from 1-2 mm small antral follicles located on the border between the ovarian cortex and medulla (FIG. 8). COCs were grouped according to the number of cumulus cell layers: 0L, 1L, or ML (FIGS. 9A, D, G). After 48 hours IVM, the percentage of oocytes from each group that were in GV, MI, or MII stages was determined (Table 5). Most of OL-COC oocytes remained in the GV stage (FIG. 9A-C). Cumulus cell expansion was observed after 24 hours' IVM in the 1L-COC and ML-COC groups (FIGS. 2E, H), and mature oocytes (MII) were seen after 48 hours (FIGS. 9F, I). Significantly more oocytes from the ML-COC group resumed meiosis and progressed MII stage after 48 hours IVM (42%) compared with oocytes from the 0L-COC and 1L-COC groups (3% and 23%, respectively; Table 5).

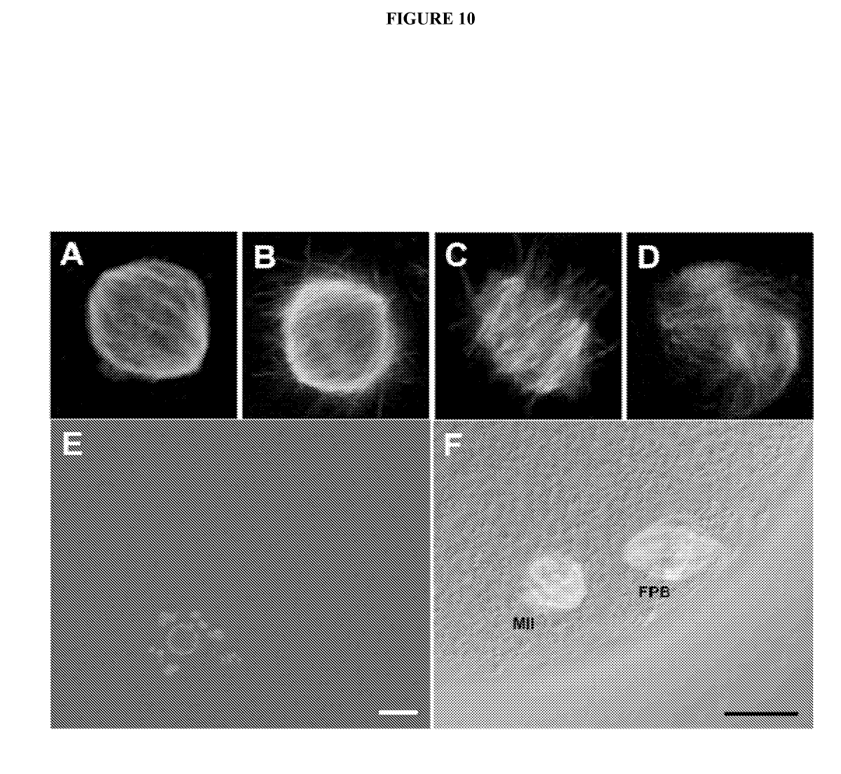

Spindle morphology and chromosome alignment in in vitro matured oocytes from small antral follicle COCs was assessed using a previously described classification system (De Santis et al. Hum Reprod, 2007, 2776-83). FIG. 10 shows representative images of in vitro matured oocytes from the 1L-COC (FIG. 10A-D) and ML-COC (FIG. 10E-H) for each of the 4 classifications of nuclear status: (A) Bipolar spindle/aligned chromosomes showing bipolar organization with pointed or flattened poles, microtubules converging at both poles, and all chromosomes present and evenly aligned at the equatorial plate (FIGS. 10A, E); (B) Bipolar spindle/nonaligned chromosomes showing bipolar organization with pointed or flattened poles and microtubules meeting at both poles, but with chromosomes with varying degrees of misalignment (FIGS. 10B, F); (C) Disarranged spindle/aligned chromosomes showing clusters of disorganized microtubules, multipolar spindles, or spindles with microtubules not converging at one or both poles, and with chromosomes associated with microtubules and closely aligned (FIGS. 10C, G); and (D) Severely disarranged or absent spindle/dispersed or absent chromosomes (FIGS. 10D, H). No statistically significant differences were found between the 1L-COC and ML-COC groups with regard to the percentage of oocytes in each of the 4 categories (Table 5). There were insufficient numbers of MII oocytes following IVM of 0L-COC oocytes to permit evaluation of nuclear status in this group.

Fertilization of In Vitro Matured Oocytes from Small Antral Follicle COCs

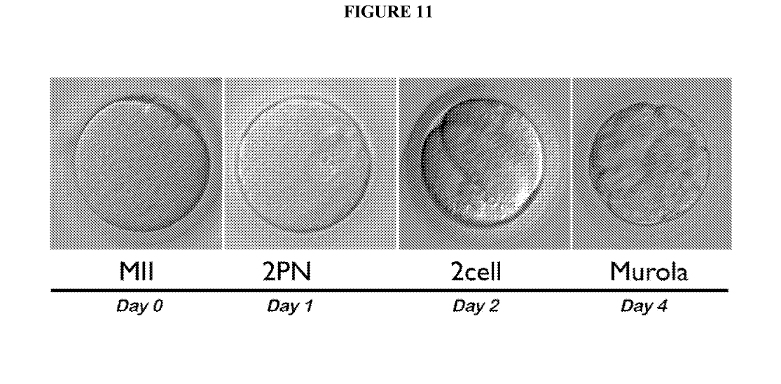

Of the 33 mature oocytes fertilized by ICSI, 8 demonstrated normal fertilization with two pronuclei. Four 2-cell embryos were obtained on day 2, and of these, 2 developed to the morula stage by day 4 (FIG. 11).

### In Vitro Growth and Maturation of Preantral Follicles

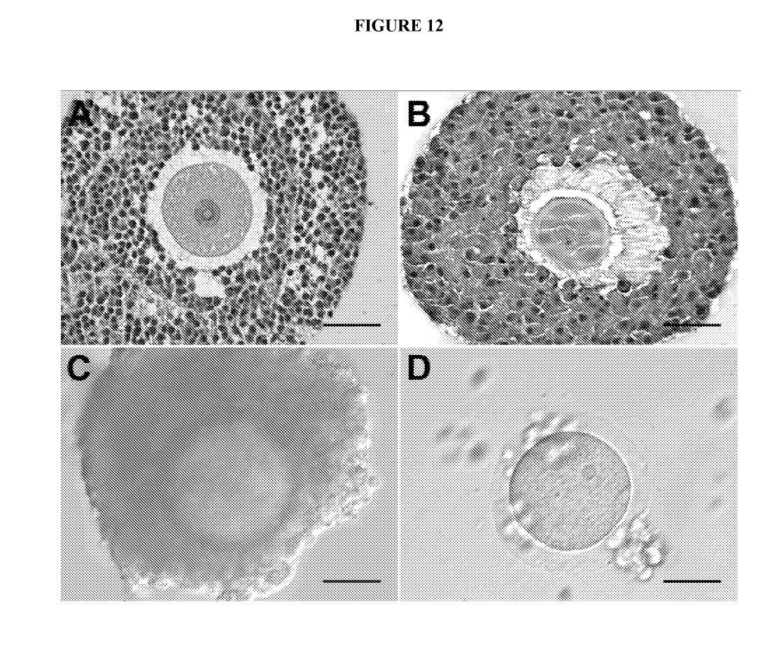

A total of 46 preantral follicles were encapsulated in FAM and cultured for 10 days in media supplemented with 0, 10, or 100 mIU/ml FSH (Table 4). The overall survival rates of follicles among the 3 groups were comparable. Follicles exhibited FSH dose-dependent increases in follicle size. However, exogenous FSH negatively impacted follicle health (FIG. 12); in the presence of 100 mIU/ml FSH, granulosa cells became hypotrophic and the oocyte lost intercellular connection with the surrounding cumulus cells (FIG. 12B). After 10 days of culture COCs were recovered from follicle. All oocytes from the FSH-10 and FSH-100 culture groups were already denuded (FIG. 12D), whereas oocytes cultured in the absence of FSH had at least one layer of cumulus cells (FIG. 12C). In addition, oocyte size was negatively correlated with FSH dose in culture (Table 7). Although IVM was performed on all of the GV-intact oocytes recovered from the FAM culture beads, none of the oocytes resumed meiosis after 48 hours of IVM.

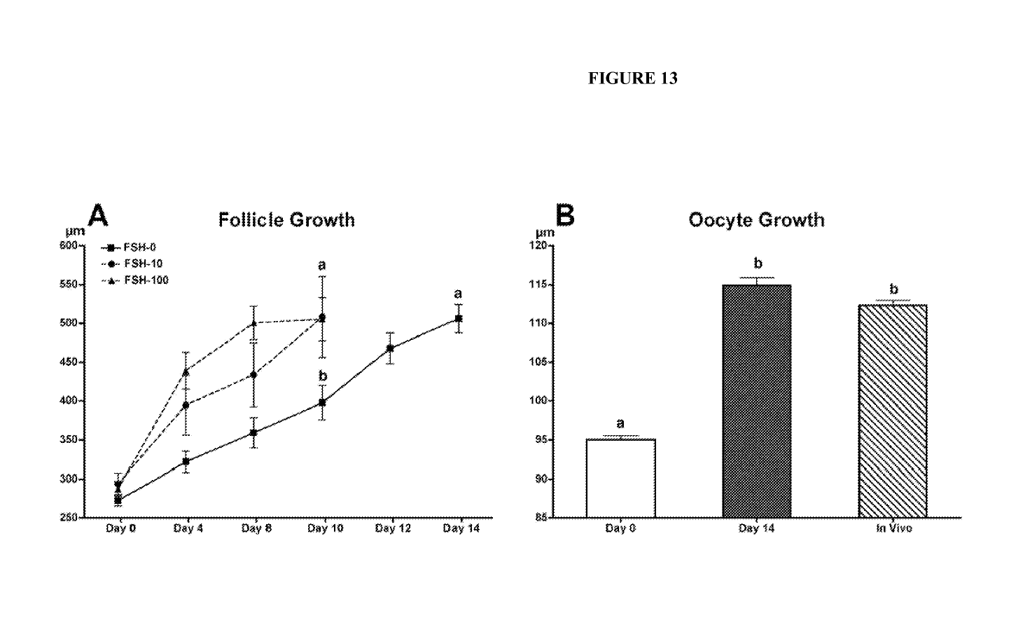

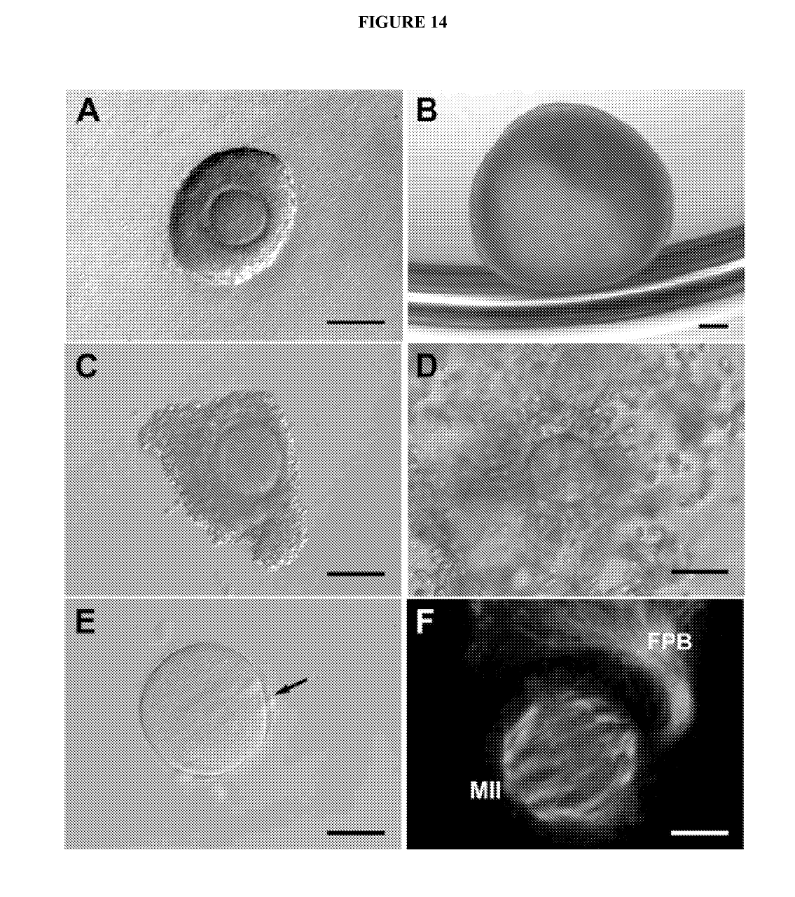

Based on these results, 31 preantral follicles were encapsulated (FIG. 14A) and cultured for 14 days in the absence of FSH. With the additional 4 days of culture, the follicles grew to diameters equal to those of follicles that has been cultured in the presence of FSH for 10 days (FIG. 13A), and more than 50% of the follicles (17 out of 31) developed to the antral stage (FIG. 14B). GV-stage oocytes grew in culture to sizes comparable to those of oocytes within small antral follicle COCs (FIG. 13B). IVM was then carried out with 16 of the COCs recovered from the FAM culture beads; the COCs had at least 1 complete layer of cumulus cells (FIG. 14C). Within 24 hours, the cumulus cells of all of in vitro matured COCs had expanded (FIG. 14D), 13 of the 16 oocytes exhibited GVBD, and 2 MII oocytes were obtained (FIG. 14E) with normal spindle structure and chromosome alignment (FIG. 14F).

TABLE 4
Baboon information and experimental design.

| Baboon | Age (y) | DPO | Small antral COCs | Preantral follicles |
|---|---|---|---|---|
| 1 | 16 | 13 | IVM + Confocal | IVFC in FSH-0, 10, 100 |
| 2 | 9 | 8 | | for 10 days |
| 3 | 10 | 9 | | |
| 4 | 11 | 7 | IVM + ICSI | |
| 5 | 16 | 9 | | IVFC in FSH-0 for |
| 6 | 11 | 3 | | 14 days |

DPO, day post ovulation;
ICSI, intracytoplasmic sperm injection;
IVM, in vitro maturation;
IVFC, in vitro follicle culture.

TABLE 5
Small antral follicle COC oocyte status at baseline and after 48 hours' IVM.

| | | Baseline | | | | | |
|---|---|---|---|---|---|---|---|
| | | Diameter | | 48 hours' IVM | | | |
| Group | N | (μm) * | SN | GV (%) | MI (%) | MII (%) | DG (%) |
| 0L-COC | 66 | 99.7 ± 0.8^{a} | | 93.3 ± 6.7^{a} | 2.9 ± 2.9^{a} | 2.9 ± 2.9^{a} | 1.0 ± 0.9 |
| 1L-COC | 112 | 102.6 ± 0.7^{b} | | 70.2 ± 7.9^{a} | 7.0 ± 7.0^{a} | 22.8 ± 7.4^{b} | 0.0 ± 0.0 |
| ML-COC | 193 | 104.6 ± 1.0^{b} | | 50.8 ± 4.9^{b} | 2.1 ± 1.4^{a} | 41.6 ± 6.1^{c} | 5.5 ± 4.8 |

Values are the average ± SEM from 6 independent experiments. Different letters within each column indicate statistically significant differences (P < 0.05).
N, total number of oocytes;
GV, germinal vesicle;
MI, metaphase I;
MII, metaphase II;
DG, degenerated.

TABLE 6
Spindle morphology and chromosome alignment in oocytes from small
antral follicle COCs after 48 hours of IVM.

Spindle morphology and
chromosome alignment

| | N | A | B | C | D |
|---|---|---|---|---|---|
| 0L-COC | 3 | Not | | | |
| | | Analyzed | | | |
| 1L-COC | 17 | 71.4% | 14.3% | 7.1% | 7.1% |
| ML-COC | 26 | 71.4% | 9.5% | 9.5% | 9.5% |

N, oocyte number.
Note that only oocytes that contained spindles with good 3D reconstruction were included in the analysis to confirm chromosome alignment.
Spindle morphology and chromosome alignment classification system has been described by De Santis, et al. 2007).

TABLE 7
Results of in vitro culture of baboon preantral follicles in FAM matrix.

| | | | Follicle Diameter | | | | Oocyte Diameter | |
|---|---|---|---|---|---|---|---|---|
| | | Survival | (μm)* | | | | (μm)^{†} | |
| Group | N | (%) | Day 0 | Day 4 | Day 8 | Day 10 | Day 0 | Day 10 |
| FSH-0 | 16 | 73.6 | 283.0 ± 11.6 | 304.1 ± 13.5^{a} | 359.2 ± 30.8^{a} | 398.0 ± 35.0^{a} | 93.5 ± 3.8 | 112.0 ± 6.7^{a} |
| FSH-10 | 16 | 84.0 | 293.9 ± 13.6 | 394.8 ± 38.7^{b} | 434.0 ± 41.3^{b} | 508.5 ± 52.2^{b} | 94.6 ± 3.0 | 101.8 ± 6.7^{b} |
| FSH-100 | 14 | 78.0 | 287.7 ± 9.4 | 439.0 ± 23.7^{b} | 500.8 ± 21.5^{c} | 505.6 ± 17.8^{b} | 92.9 ± 8.2 | 98.4 ± 2.9^{c} |

Different letters within each column indicate statistically significant differences (P < 0.05).
N, starting follicle number.
*Values are the average ± SEM;
^{†}Values are the average ± SD.

### Example 3

### Tissue-Engineered Advance in the In Vitro Two-Step Culture of Early Stage Ovarian Follicles in Mouse

To develop an in vitro strategy to support the growth of early-stage follicles and produce mature oocytes competent for fertilization, whole ovaries from 8-day-old mice were cultured for 4 days, and then secondary follicles were isolated and cultured for 12 days in a three-dimensional alginate or fibrin-alginate (FA) hydrogel matrix. Analyses of outcomes included histologic evaluation of follicle development, steroid hormone production, and rates of oocyte maturation, oocyte fertilization, and embryo formation.

### A. Materials and Methods

### Animals

C57BL/6j×CBA/Ca F1 hybrid mice study were housed and bred for the purposes of the study. Eight-day-old F1 female mice were used in this study. All animals were housed in a temperature- and light-controlled environment (12L:12D) and were provided with food and water ad libidum.

### Organ Culture of 8-Day-Old Mouse Ovaries

As reported previously (Nilsson et al. (2007) Reproduction 134:209-221; herein incorporated by reference in its entirety), ovaries were excised from the ovarian bursa and washed twice with culture medium: α-minimal essential medium (αMEM) supplemented with recombinant FSH (10 mIU/mL; A. F. Parlow, National Hormone and Peptide Program, National Institute of Diabetes and Digestive and Kidney Diseases), bovine serum albumin (3 mg/mL), bovine fetuin (1 mg/mL; Sigma-Aldrich, St. Louis, Mo.), insulin (5 ng/mL), transferrin (5 ng/mL), and selenium (5 ng/mL). Ovaries were transferred into 24-well plates with tissue culture well inserts (nontissue culture treated; Millicell-CM, 0.4-um pore size; Millipore Corp., Billerica, Mass.). Approximately 400 μL of culture medium was added to the compartment below the membrane insert, such that ovaries on the membrane were covered with a thin film of medium. Up to six ovaries were placed in each well. The ovaries were incubated at 37° C., 5% CO₂, for 4 days. Every other day, 150 μL of media was replaced with fresh culture media.

### Histologic Analysis and Follicle Classifications

Ovaries from 8- and 12-day-old mice were fixed overnight in a 4% paraformaldehyde solution at 4° C. and then dehydrated in an ethanol series and embedded in paraffin wax. Sections (5 μm) were stained with hematoxylin and eosin (H&E). The number of follicles at each developmental stage was counted and averaged in three serial sections from the largest cross-sections through the center of the ovary (Nilsson et al. (2007) Reproduction 134:209-221; Chen et al. (2007) Endocrinol. 148:3580-3590; each herein incorporated by reference in its entirety). Only follicles that contained an oocyte nucleus were counted. Follicles were classified as primordial (stage 0), primary (stage 1), and secondary (stage 2) as previously described (Yan et al. (2008) Biol. Reprod. 278:1153-1161; herein incorporated by reference in its entirety). Follicle counting results were calculated as percentages to account for differences between preculture and postculture ovaries.

### Alginate Hydrogel and Fibrin-Alginate Gel Preparation

Alginate hydrogel was prepared as described previously (West et al. (2007) Biomaterials 28:4439-4448; Xu et al. (2006) Tissue Eng. 12:2739-2746; each herein incorporated by reference in its entirety). FA gel was prepared as described (Shikanov et al. (2009) Biomaterials 30:5476-5485; herein incorporated by reference in its entirety; and Example 1). Tisseel® fibrin sealant kits (Baxter, Deerfield, Ill.) were used according to the kit instructions. Fibrinogen and thrombin were reconstituted with aprotinin (3000 KIU/mL) solution and calcium chloride (40 mM) separately. Appropriate concentrations of both solutions were attained by dilution in tris-buffered saline (TBS) solution. The FA gel was prepared by mixing 50 mg/mL fibrinogen solution with 0.5% alginate solution at 1:1 and then adding the same volume of 50 IU/mL thrombin solution to the mixture.

### Isolation, Encapsulation and Culture In Vitro of Preantral Follicles

After 4 days of ovary tissue culture, secondary follicles were mechanically isolated using insulin-gauge needles and placed into L15 media (Invitrogen, Carlsbad, Calif.) with 1% fetal calf serum (FCS), then transferred into αMEM supplemented with 1% FCS and incubated at 37° C., 5% CO₂, for 2 hours. Follicles with centrally located oocytes and at least two layers of granulosa cells were encapsulated into alginate beads (0.25% [w/v]) or FA beads (0.125% alginate, 12.5 mg/mL fibrinogen) (Xu et al. (2006) Biol. Reprod. 75:916-923; herein incorporated by reference in its entirety). Encapsulation in FA beads was performed as described (Shikanov et al. (2009) Biomaterials 30:5476-5485; herein incorporated by reference in its entirety; and Example 1). Alginate and FA beads containing follicles were washed twice in culture media. One bead was placed in each well of a 96-well plate, in 100 μL culture media and incubated at 37° C., 5% CO₂, for 12 days. Every other day, 50 μL of the media was replaced by fresh culture media, and follicle survival and diameter were assessed as described previously (Xu et al. (2006) Biol. Reprod. 75:916-923; herein incorporated by reference in its entirety). At the end of the culture period, the media was replaced by L15 medium (100 μL) containing alginate lyase (10 units/mL; Sigma-Aldrich), and the beads were incubated for 30 minutes at 37° C. Follicles were then removed from the degraded alginate bead by mechanical isolation (Xu et al. (2006) Tissue Eng. 12:2739-2746; Xu et al. (2006) Biol. Reprod. 75:916-923; each herein incorporated by reference in its entirety).

### In Vitro Maturation and Fertilization of Oocytes

Cumulus-enclosed oocytes (CEOs) were collected from antral follicles released from alginate or FA beads. The CEOs were placed in αMEM, 10% FCS, 1.5 IU/mL hCG, and 5 ng/mL epidermal growth factor (EGF; Sigma-Aldrich) for 18 hours at 37° C., 5% CO₂ (des Rieux et al. (2009) J. Control. Release 136:148-154; herein incorporated by reference in its entirety).

Sperm was collected from the cauda epididymis of proven CD-1 male breeder mice using Percoll gradient centrifugation as described previously (Xu et al. (2006) Tissue Eng. 12:2739-2746; herein incorporated by reference in its entirety). The sperm was capacitated for 30 minutes in IVF media (KSOM; Specialty Media, Phillipsburg, N.J.) containing 3 mg/mL bovine serum albumin and 5.36 mM D-glucose. Approximately 5-10 metaphase II (MII)-stage oocytes were placed in a 100-4, droplet of IVF medium containing sperm, placed under mineral oil, and incubated for 7-8 hours at 37° C., 5% CO₂. Fertilized oocytes were washed three times in fresh KSOM to remove all sperm and then transferred into a 50-μL fresh KSOM microdrop under mineral oil overnight. Embryos that cleaved to the two-cell stage were recorded as fertilized (Liu et al. (2001) Biol. Reprod. 64:171-178; Xu et al. (2006) Biol. Reprod. 75:916-923; each herein incorporated by reference in its entirety).

### Hormone Assays

E₂ and P were measured in conditioned media collected on follicle culture days 2, 6, and 12. Conditioned media from each time point were pooled together, and the average concentration at each time point was determined from three independent experiments. All measurements were performed by electrochemoluminescent assay using an Immulite 2000 Analyzer (Roche, Indianapolis, Ind.). Interassay variations were 6.1% for E₂ and 5.4% for P, and the limits of sensitivity were 5 pg/mL for E₂ and 0.03 ng/mL for P.

### Statistical Analysis

All experiments were performed at least three times. Values are given as mean±SEM, and statistical analysis was done using Student's t test. Differences were considered significant at P <0.05.

### B. Results

### Follicle Development in Organ Culture

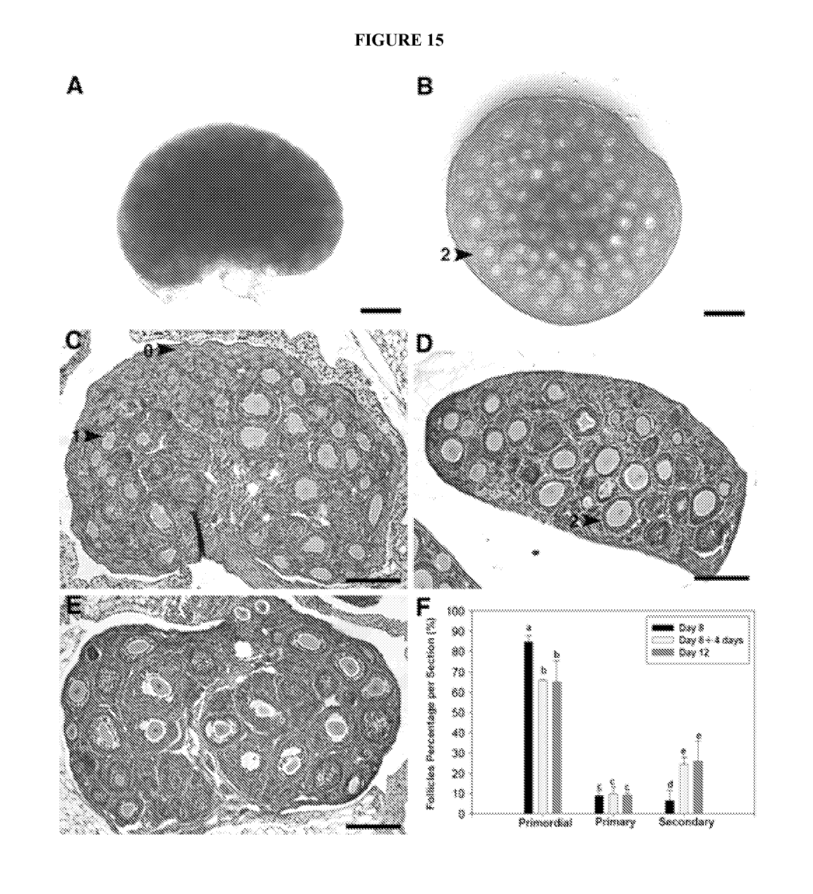

Ovaries from 8-day-old mice contained mostly primordial follicles (84.8±3.2%), with a few primary (8.8±2.5%) and secondary follicles (6.4±5.2%; FIGS. 15A, 15C, 15F). After 4 days of organ culture in vitro (FIGS. 15B, 15D), primordial follicles represented a smaller percentage of the total follicle pool (65.7±0.5%), similar to the follicle distribution seen in ovaries from 12-day-old mice (65±10.6%; FIGS. 15E, 15F). The proportion of secondary follicles increased significantly during the 4-day culture, from 6.4±5.2% to 24.5±3.3% (P <0.05; FIGS. 15A vs. 15B; FIGS. 15C vs. 15D). The ratio of activated follicles in the cultured ovaries was similar to that of 12-day-old ovaries (25.8±10%). There were no differences in the proportion of primary follicles in the 8-day-old ovaries before or after culture and in the 12-day old ovaries (FIG. 15F).

### Secondary Follicle Growth in the Alginate and Fibrin-Alginate Culture Systems

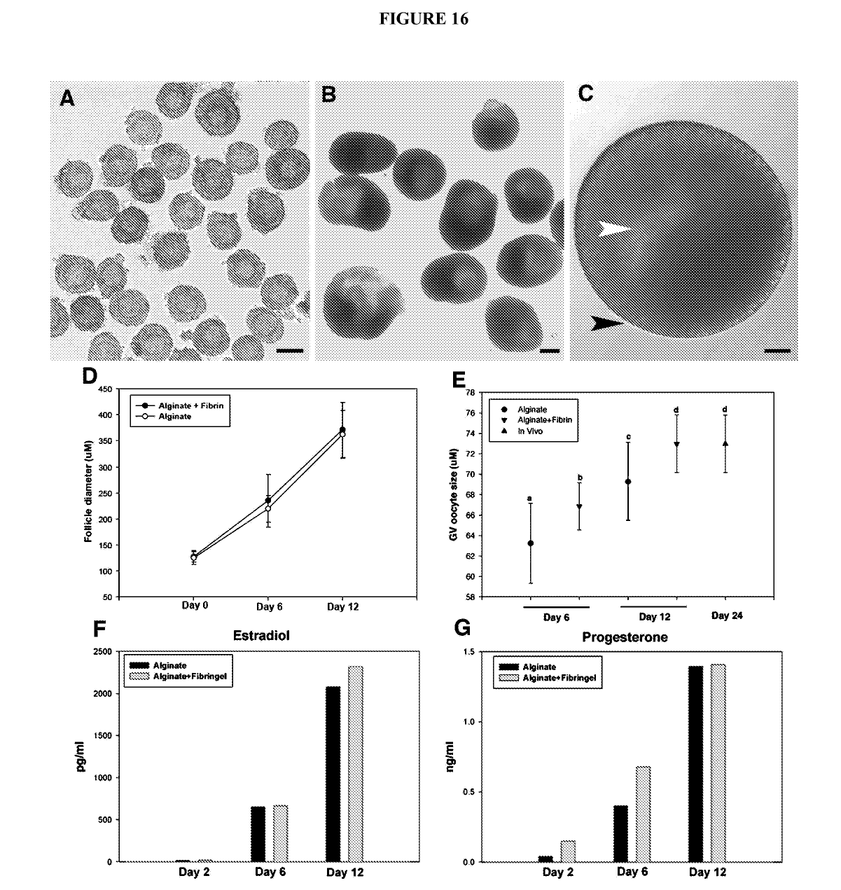

A total of 430 secondary follicles, with a diameter range of 111-137 μm, were isolated from the cultured ovaries (FIG. 16A), embedded in FA beads or alginate beads, and cultured for 12 days (FIG. 16B). At day 12, the majority of follicles had survived the culture period in either FA beads (74.8±4.6%) or alginate beads (68.6±5.5%; Table 8). Antrum formation and the appearance of a laminar-like theca cell layer were seen more frequently in follicles cultured in the FA system compared with follicles cultured in alginate (antrum, 72.0±3.9% vs. 59.7±5.6% (P <0.05); theca layer, 72.3±3.2% vs. 64.7±4.6% (P<0.05); FIG. 16C; Table 8). Follicle diameter increased significantly, from 124±2.2 μm at day 0 to 362.4±10.1 μm in alginate and 371.6±8.8 μm in FA (FIG. 16D). Follicle-enclosed oocytes in both groups also increased in size during the 12-day culture (FIG. 16E); however, final oocyte diameter was larger in the FA-cultured follicles compared with alginate-cultured follicles (73±0.6 μm vs. 69.3±0.7 μm; P<0.05). By comparison, the average oocyte diameter in secondary follicles from 24-day-old mice was 73±0.6 μm (FIG. 16E). As shown in FIGS. 16F and 16G, the secretion patterns of E₂ and P were consistent with observed changes in follicle morphology and cell differentiation in the cultured follicles. During the first 6 days of culture, both E₂ and P levels rose more slowly than during the last 6 days of culture. There was no significant difference in steroid secretion between follicles cultured in alginate or FA.

TABLE 8
Assessment parameters of follicle and oocyte growth cultured in two
different gels.

| | | | Theca | | | | | | | Two-cell |
|---|---|---|---|---|---|---|---|---|---|---|
| | | Survival | Layer | Antrum | | DG | GV | GVBD | MII^{c} | embryos^{d} |
| Group | N^{a} | (%) | (%) | (%) | n^{b} | (%) | (%) | (%) | (%) | (%) |
| Alginate | 230 | 68.6 ± 5.5 | 64.7 ± 4.6^{e} | 59.7 ± 5.6^{e} | 96 | 11.3 ± 1.3 | 13.7 ± 1.9 | 75 ± 0.6^{e} | 61.3 ± 2.4^{e} | 33 ± 1.7^{e} |
| Fibrin + | 200 | 74.8 ± 4.6 | 72.3 ± 3.2^{f} | 72.0 ± 3.9^{f} | 50 | 7.7 ± 1.2 | 6 ± 0.6 | 86.3 ± 0.9^{f} | 88 ± 8.7^{f} | 54 ± 4.0^{f} |
| Alginate | | | | | | | | | | |

Note:
Values are the average ± SEM of multiple follicles or oocytes from at least three independent cultures;
GV, germinal vesicle;
GVBD, germinal vesicle breakdown;
MII, metaphase II;
DG, degenerate.
^{a}N = number of secondary follicles.
^{b}n = number of CEOs from antral follicles.
^{c}The percentage of MII oocyte was calculated as a proportion of oocytes undergoing GVBD.
^{d}Two-cell embryos/MII oocytes.
^{e,f}Different superscripts with each column indicate statistically significant differences (p < 0.05).

### Oocyte Meiosis and Fertilization Competence

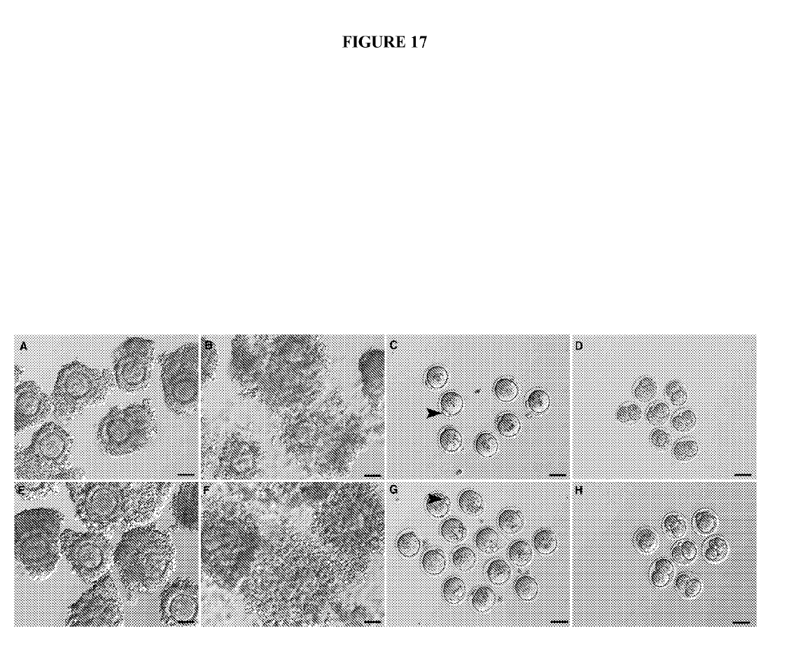

CEOs (n=96 from alginate-cultured follicles (FIG. 17A), and n=50 from FA-cultured follicles (FIG. 17E) were stimulated with hCG and EGF for 18 hours. After treatment, significant cumulus cell expansion was observed in both groups (FIGS. 17B, 17F). Most of the oocytes in both groups resumed meiosis, underwent germinal vesicle breakdown (GVBD), and matured to MII with extrusion of a first polar body (FIGS. 17C, 17G; Table 8). In the FA-cultured group, 86±0.9% of the oocytes progressed to MI, compared with 75±0.6% in the alginate-cultured group (P<0.05). Moreover, the percentage of oocytes that reached MII was higher in the FA-cultured group than in the alginate-cultured group (88±8.7% vs. 61.3±2.4%; P <0.05; Table 8). In the alginate-cultured group, 33±1.7% of the MII oocytes could be fertilized and cleaved to two-cell embryos, whereas in the FA-cultured group, 54±4% of MII oocytes formed two-cell embryos (P<0.05; FIGS. 17D vs. 17H; Table 8).

While the present invention is not limited to any particular mechanism, and an understanding of the mechanism is not necessary to practice the present invention, it is contemplated that key insights into early follicle growth and its effect on later oocyte maturation gained during experiments described herein. Improved methods of the present invention find use in clinical application in the preservation of fertility (Xu et al. (2006) Biol. Reprod. 75:916-923; herein incorporated by reference in its entirety).

Organ culture maintains the in vivo microenvironment of the follicles, including the surrounding stromal cells and their intercommunication with early-stage follicles, and the connectivity between cellular compartments within the follicle. The growth of primordial and primary follicles in vitro has been accomplished using organ culture (O'Brian et al. (2003) Biol. Reprod. 68:1682-1686; Wandji et al. (1996) Biol. Reprod. 55:942-948; Wandji et al. (1997) Hum. Reprod. 12:1993-2001; Hovatta et al. (1997) Human Reprod. 12:1032-1036; each herein incorporated by reference in its entirety), but the efficiency of reported methods has been low (Abir et al. (1999) Human Reprod. 14:1299-1301; Hovatta et al. (2000) Mol. Cell Endocrinol. 169:95-97; each herein incorporated by reference in its entirety). In experiments conducted during the course of developing some embodiments of the present invention, a two-step culture system was developed that combined early follicle growth within the intact ovary with a hydrogel-based follicle culture system to support the further growth and development of secondary follicles. A follicle survival rate of 68% and an antrum formation rate of 59% was achieved using an alginate-based, 3D follicle culture system. Higher rates of follicle survival (74%) and antrum formation (72%) using an FA culture system.

While the present invention is not limited to any particular mechanism, and an understanding of the mechanism is not necessary to practice the present invention, it is contemplated that the development of a culture system that supports follicle growth and oocyte maturation beginning at the early follicle stage allows access to a significantly greater number of follicles for in vitro maturation and IVF. Herein a two-step protocol is described that combines traditional organ culture and a novel hydrogel-based 3D follicle culture technique. Whole ovaries from 8-day-old mice, which contained primarily primordial follicles with a few primary and secondary follicles, were cultured to support early-stage follicle growth and development into the secondary follicle stage. While the present invention is not limited to any particular mechanism, and an understanding of the mechanism is not necessary to practice the present invention, it is contemplated that the cultured ovary acts as an incubator, where important stroma-cell and cell-cell interactions remain intact, and the presence of local paracrine and autocrine factors support primordial and primary follicle growth. With 4-day culture of 8-day-old mouse ovaries, it was possible to achieve a similar degree of early-stage follicle development and transition to secondary follicles as in 12-day-old ovaries. In the second step, secondary follicles were isolated from the cultured ovaries and grown in alginate beads for 12 days to support further follicle development (Kreeger et al. (2006) Biomaterials 27:714-723; Xu et al. (2009) Biotechnol. Bioeng. 103:378-386; Xu et al. (2006) Tissue Eng. 12:2739-2746; Xu et al. (2006) Biol. Reprod. 75:916-923; Shikanov et al. (2009) Biomaterils 30:5476-5485; each herein incorporated by reference in its entirety; and Example 1). During this time, follicles significantly increased in mean diameter, with formation of an antral cavity and proliferation and differentiation of granulosa cells and theca cells. The mean diameter of oocytes also increased and cumulus cells expanded significantly in response to hCG. The majority of oocytes resumed meiosis and were competent to undergo GVBD and polar body extrusion, and fertilized oocytes developed to two-cell embryos. The ability to produce embryos starting from early-stage follicles from 8-day-old mice was demonstrated.

FA hydrogel was superior to alginate in regard to follicle growth and differentiation, thus producing a larger percentage of oocytes competent for fertilization and a greater number of two-cell embryos than alginate alone. Studies have shown that the efficiency of producing fertilizable oocytes in vitro is influenced by many factors, leading to unpredictability (Eppig et al. (1989) Biol. Reprod. 41:268-246; Wang et al. (2007) Reprod. Fertil. Dev. 19:1-12; each herein incorporated by reference in its entirety). Fibrin is naturally derived protein, and commercial fibrin consists of thrombin and fibrinogen that is cryoprecipitated from blood plasma, as well as small amounts of fibronectin, transforming growth factor-1, basic fibroblast growth factor, epidermal growth factor, vascular endothelial growth factor, and other biomolecules (de Rieux et al. (2009) J. Control Release 136:148-154; herein incorporated by reference in its entirety). Some of these factors play an important role in follicle development (Knight et al. (2006) Reproduction 132:191-206; herein incorporated by reference in its entirety), and fibrin itself supports a number of cellular processes, including growth, proliferation, and differentiation (de Rieux et al. (2009) J. Control Release 136:148-154; herein incorporated by reference in its entirety). The FA hydrogel also has unique dynamic mechanical properties, as cell-secreted proteases degrade the fibrin in the surrounding bead and remodel the local environment. Alginate is produced by brown algae and permits diffusion of hormones and other molecules from the surrounding environment (West et al. (2007) Semin. Reprod. Med. 25:287-299; herein incorporated by reference in its entirety). Thus, the combination of alginate and fibrin maintains the 3D architecture of follicles and provides an environment that supports follicle growth.

### Example 4

### Aprotinin as an Inhibitor of Fibrin Degradation

This example describes the effect of aprotinin addition on follicle culture.

### A. Materials and Methods

### Animals and Materials

Two-layered secondary follicles were mechanically isolated from 12-day-old female F1 hybrids (C57BL/6j×CBA/Ca). Animals were purchased (Harlan, Indianapolis, Ind.), housed in a temperature and light controlled environment (12 L: 12 D) and provided with food and water ad libidum. Animals were fed Teklad Global irradiated 2919 chow, which does not contain soybean or alfalfa meal and therefore contains minimal phytoestrogens. Unless otherwise noted, all chemicals were purchased from Sigma-Aldrich (St. Louis, Mo.), stains and antibodies from MolecularProbes (Eugene, Oreg.), and media formulations from Invitrogen (Carlsbad, Calif.). Sodium alginate (55-65% guluronic acid) was provided by FMC BioPolymers (Philadelphia, Pa.) and Tisseel® substrate (Baxter Healthcare, BioScience Division, Westlake village, Calif.) was used for fibrin gels preparation.

### FA-IPN Preparation

The fibrinogen-containing component of Tisseel® substrate was reconstituted in aprotinin (3000 KIU/mL) solution and the thrombin component was reconstituted in 30 mM CaCl₂, according to the Baxter kit instructions. Both solutions were diluted to the appropriate concentrations by diluting the fibrinogen containing component in Tris-buffered saline solution (TBS) and thrombin in 30 mM CaCl₂ in TBS. Alginate aliquots were prepared as previously described (Kreeger, 2006) and diluted to 0.5% w/v. IPNs were prepared by mixing fibrinogen solution (50 mg/mL) with alginate solution 0.5% in 1:1 ratio, and then adding thrombin solutions of 50 IU/mL to the mixture at 1:1 ratio. The fibrinogen-alginate mix and the thrombin solutions were filled with equal volumes in 1 mL syringes and injected using the Duploject System provided with the kit, while mixing in the needle. Thus, the final concentrations in the gels of fibrinogen and alginate were 12.5 mg/mL and 0.125%, respectively.

### Follicle Isolation, Encapsulation and Culture

Two-layered secondary follicles (100-130 μm, type 4) were mechanically isolated as described before (West, Xu) and encapsulated in FA-IPNs or fibrin gels. Fibrinogen-alginate solution (7.5 μL, 25 mg/mL fibrinogen, 0.25% alginate) were pipette on alcohol wiped glass slide with 3 mm spacers and covered with parafilm and individual follicles were pipette into the droplets. Thrombin solutions (7.54 of 50 IU/mL) were pipetted on top of each droplet with the follicle, covered with the second glass slide covered with alcohol wiped parafilm and transferred to the 37° C. incubator for 5 min. The beads with the follicles were washed in maintenance media (αMEM, 1 mg/mL bovine serum albumin and penicillin-streptomycin) and transferred to 96-well plates with 150 μl of culture media (αMEM, 3 mg/mL bovine serum albumin (MP Biomedicals, Inc., SOLON, Ohio), 10 mIU/mL rFSH, 1 mg/mL bovine fetuin, 5 μg/mL insulin, 5 μg/mL transferrin and 5 ng/mL selenium). Encapsulated follicles were cultured at 37° C. in 5% CO₂ for 12 days. Every other day, half of the media (75 μL) was exchanged and stored at −80° C. for steroid assay. Two aprotinin concentrations were tested: 0.01 and 0.1 TIU/mL. The aprotinin was added to the culture media on days 0, 2 and 4.Starting at day 6 of the culture follicles were grown in aprotinin-free media. Follicle survival and diameter were assessed using an inverted Leica DM IRB microscope with transmitted light (Leica, Bannockburn). The diameter of follicles containing oocytes was measured in duplicate from the outer layer of theca cells using ImageJ 1.33U and based on a calibrated ocular micrometer.

### Oocyte Meiotic Competence

Oocyte meiotic competence was assessed by maturation after 12 days of culture. Follicles were removed from the beads by 10 min incubation of the beads in a 10 IU/mL solution of alginate lyase, which enzymatically degrades the alginate, in prewarmed L-15 media. Antral follicles were transferred to αMEM containing 10% FBS, 5 ng/mL epidermal growth factor and 1.5 IU/mL human chorionic gonadotropin and were matured at 37° C. n 5% CO₂ for 14-16 h. Oocytes were denuded then from the surrounding cumulus cells by treating with 0.3% hyaluronidase. Oocyte state was assessed from the light microscopy images, and characterized as follows: germinal vesicle breakdown (GVBD) if the germinal vesicle was not present, GV if there was an intact germinal vesicle, metaphase II (MII) if a polar body was present in the perivitelline space and degenerated (DG) if the oocyte was fragmented or shrunken.

### B. Results

### Fibrin Degradation

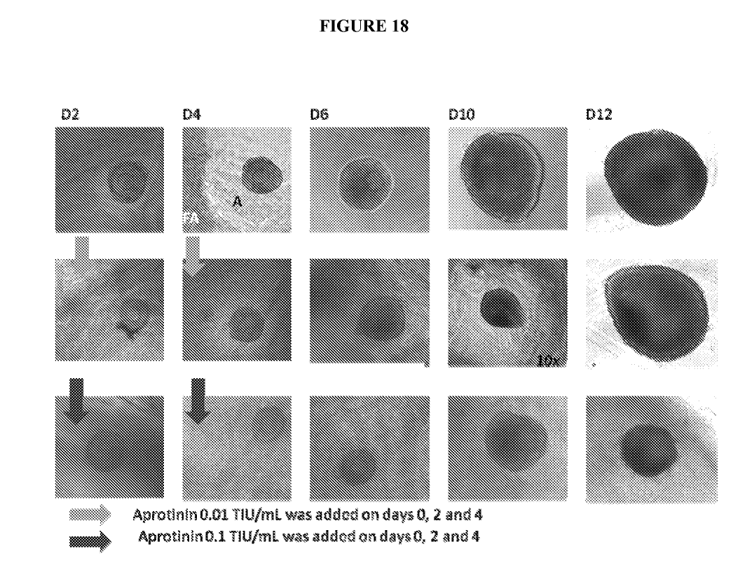

The fibrin component of the IPN gradually disappeared from the hydrogel when cultured without aprotinin, with the initial clearance occurring adjacent to the follicle and then progressing toward the edge of the hydrogel bead (FIG. 18A). On the second day of a culture, the region immediately adjacent to the follicle became clear, consistent with the degradation of the fibrin component of the IPN. When 0.01 TIU/mL aprotinin was added on days 0, 2 and 4 to the media, fibrin around the follicle did not degrade and the degradation ring around the follicle appeared only on day 6 for some extend after aprotinin removal from the culture (FIG. 18B). When greater amount of aprotinin was added to the culture (0.1 TIU/mL) fibrin degradation was completely blocked and it did not degrade even after the removal of the inhibitor from the media at day 6 (FIG. 18C).

### Two-Layered Secondary Follicle Growth

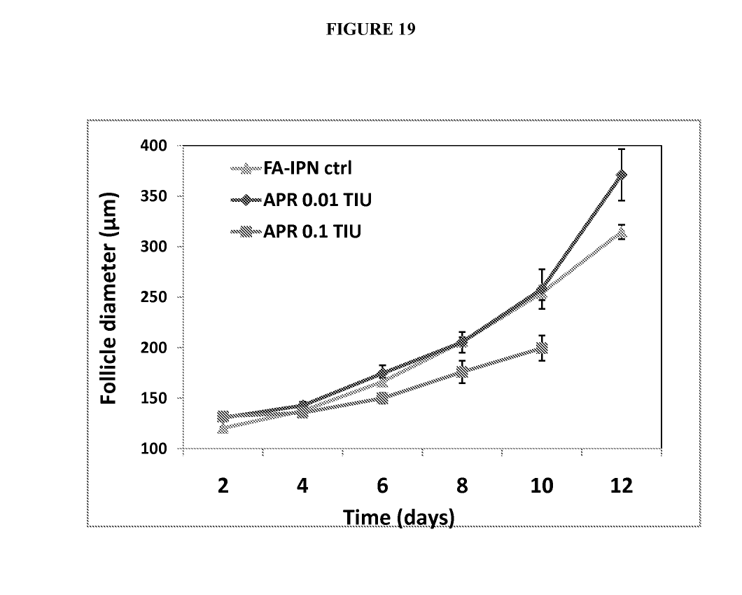

Follicle growth and size increase are summarized in FIG. 19. Follicles that were cultured in FA-IPN without aprotinin addition to the media increased in their diameter from 120 μm on day 2 to 330 μm on day 12, which is an increase of 160%. When 0.01 TIU/mL aprotinin was added to the culture media on days 0, 2 and 4 the follicle size on day 12 was greater and reached 370 μm. Addition of 0.1 TIU/mL to the culture media caused a non-reversible delay in follicle growth and follicles reached only 170 μm on day 10. A fluid filled antrum cavity was observed on day 12 for control (no aprotinin) and 0.01 TIU/mL (FIGS. 18A and 18B), which is consistent with in vivo morphology.

### Oocyte Quality

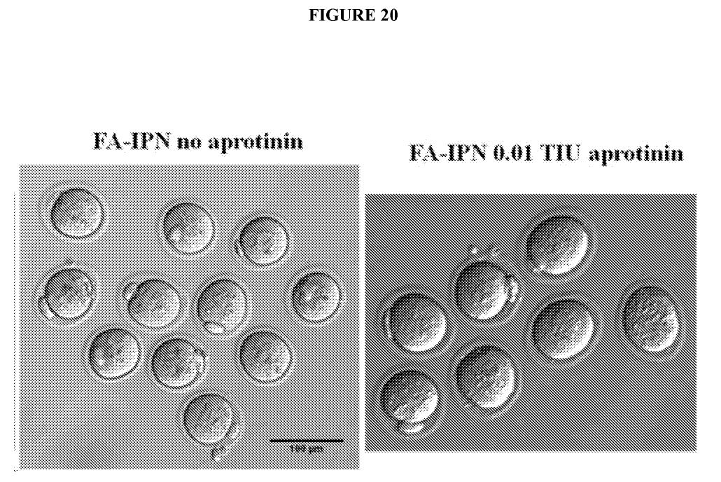

The quality of oocytes obtained from follicles cultured within FA-IPNs was subsequently measured by their ability to resume meiosis. Oocytes from follicles cultured in FA-IPN without aprotinin demonstrated high rate (82%) of Metaphase II (MII) stage and polar body extrusion, while follicles cultured with aprotinin 0.01 TIU/mL reached 88% of MII stage. Follicles cultured in high content of aprotinin were too small to mature and the extraction of the follicle from non-degraded fibrin gel required prolonged exposure to collagenase that damaged follicles and oocytes. FIG. 20 demonstrates oocytes from control and low aprotinin conditions that extruded the first polar body and exhibited a normal MII configuration.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in cell biology, reproductive biology, or related fields are intended to be within the scope of the following claims.

1. A method of culturing an organized cell cluster in vitro comprising:
a) encapsulating an organized cell cluster in a two-component interpenetrating network (IPN); and
b) culturing said encapsulated organized cell cluster in vitro.

2. The method of claim 1, wherein said two-component interpenetrating network comprises fibrin and alginate.

3. The method of claim 1, wherein said organized cell cluster is selected from the group consisting of an ovarian follicle, matrix-directed cardioprogenitor cells, embryoid bodies, and primary cell co-cultures.

4. The method of claim 3, wherein said organized cell cluster is an ovarian follicle.

5. The method of claim 4, wherein said ovarian follicle is selected from the group consisting of a primordial follicle, a primary follicle, a secondary follicle, a preantral follicle, and an antral follicle.

6. The method of claim 1, wherein said encapsulating occurs by introduction of said organized cell cluster into said two-component interpenetrating network, wherein said interpenetrating network is in a form selected from the group consisting of a bead, a culture plate insert, a transwell, and a droplet.

7. The method of claim 1, wherein said two-component interpenetrating network comprises a cross-linking agent.

8. The method of claim 7, wherein said cross-linking agent is thrombin.

9. The method of claim 1, wherein said two-component interpenetrating network comprises calcium chloride.

10. The method of claim 2, wherein said alginate is present at a final concentration of 0.125%.

11. The method of claim 2, wherein said fibrin is formed by polymerization of fibrinogen, wherein said fibrinogen is present in said interpenetrating network at a final concentration of 12.5 mg/ml.

12. The method of claim 8, wherein said thrombin is present at a final concentration selected from 5 IU/mL, 50 IU/mL, and 500 IU/mL.

13. The method of claim 1, wherein said interpenetrating network further comprises a protease inhibitor.

14. The method of claim 13, wherein said protease inhibitor is aprotinin.

15. The method of claim 4, further comprising subjecting said cultured, organized ovarian follicle to a process selected from the group consisting of in vitro maturation and in vitro fertilization.

16. The method of claim 1, wherein said interpenetrating network further comprises a proteinaceous extract of Engelbreth-Holm-Swarm mouse sarcoma.

17. The method of claim 16, wherein said extract comprises Matrigel™ matrix.

18. The method of claim 16, wherein said culturing is conducted in the presence of FSH.

19. A system for culturing an organized cell cluster in vitro, said system comprising:
i) an organized cell cluster, and
ii) a two-component interpenetrating network.

20. A kit for culturing an organized cell cluster in vitro, said kit comprising:
i) fibrinogen,
ii) alginate,
iii) thrombin, and
iv) calcium.
